(19) United States
(12) Patent Application Publication
(10) Pub. No.: US 2023/0002780 A1
(43) Pub. Date: Jan. 5, 2023

(54) EXPRESSION OF SEVERE ACUTE RESPIRATORY SYNDROME CORONAVIRUS 2 (SARS-CoV-2) SPIKE PROTEIN SEQUENCES IN PLANTS AND PLANT PRODUCED VACCINE FOR SAME

(71) Applicant: Applied Biotechnology Institute, Inc., San Luis Obispo, CA (US)

(72) Inventors: John Howard, San Luis Obispo, CA (US); Erin Egelkrout, San Luis Obispo, CA (US)

(21) Appl. No.: 17/808,901

(22) Filed: Jun. 24, 2022

Related U.S. Application Data
(60) Provisional application No. 63/202,816, filed on Jun. 25, 2021.

(51) Int. Cl. C12N 15/82; A61K 39/215

(52) U.S. Cl. CPC C12N 15/8258; A61K 39/215

(57) ABSTRACT

A plant produced vaccine for Severe Acute Respiratory Syndrome Coronavirus 2 (SARS-CoV-2) is provided where the Spike protein of the virus is expressed in a plant by introducing into a plant a construct comprising a promoter preferentially directing expression to seed of said plant, a nucleic acid encoding the Spike protein and a nucleic acid targeting expression to the endoplasmic reticulum of the plant. The plant expresses the 51 polypeptide at levels of at least 10 mg/kg of seed of said plant. When orally administered to an animal, a protective response is observed including a serum antibody response.

### CROSS-REFERENCE TO RELATED APPLICATIONS

This application claims priority under 35 U.S.C. § 119 to provisional application Ser. No. 63/202,816 filed Jun. 25, 2021. The disclosure of which is hereby incorporated in its entirety by reference.

### BACKGROUND

COVID-19 caused by Severe Acute Respiratory Syndrome Coronavirus 2 (SARS CoV-2) has demonstrated the devastating impact of a pandemic disease on the health of the global population as well as show that it can rapidly sink the global economy. During the immediate outbreak, we had to rely on isolating individuals to stop the spread of the disease. In the long-term, safe and effective vaccines have been shown to be the most cost-effective strategy to control viral diseases. During the time prior to the availability of a vaccine, the virus has had ample time to spread as it is readily transmissible by symptomatic as well as asymptomatic individuals, and it can survive in its natural form in animal reserves and therefore is likely to linger.

While a vaccine will undoubtedly be of great value, we need to have realistic expectations about what can be accomplished with traditional vaccines and how long it will take to immunize the global population. To keep this in perspective, consider that we have efficacious vaccines for many other diseases but there are still large at-risk populations that are not vaccinated due to hurdles beyond the efficacy of the vaccine itself. These include limitations due to production, distribution, administration, and cost. As an example, there has been an efficacious vaccine for hepatitis B for decades, yet 2 billion people have been infected and 700,000 people die each year³. Reducing the threat of COVID-19 with a vaccine will face the same hurdles as other vaccines and some unique to COVID-19.

Mass immunization on the scale and time frame anticipated for COVID-19 is unprecedented. The leading companies developing COVID-19 vaccines are reporting collectively produce over 1 billion doses per year. Production will be a challenge as this will be on top of other programs such as flu vaccines that require 100 million doses/year, and the supply of flu vaccines has become limiting in past years. New production facilities can be constructed but would cost several hundred million dollars and take 3-4 years to build⁵. However, assuming 2 doses are required and using these optimistic production estimates, it will take years to immunize the world population.

The cost of a COVID-19 vaccine has been estimated at $10-$150 per dose with a likely target of $40. There are also the added expenses of administration by skilled medical personnel and the need for the cold chain. As is the case for many other current vaccines, these cost constraints result in preventable diseases going unchecked and inflict high levels of morbidity and mortality on large populations.

Some highly efficacious commercial vaccines such as hepatitis B have over 85% efficacy, while others such as flu vaccines are typically closer to 50% or even lower. Lack of efficacy is an obvious problem in all cases but, in even the best-case scenario, it can still leave millions of people vulnerable. Poor responders to vaccines include groups with predisposed medical conditions such as the elderly, obese, diabetics and immunocompromised⁹⁻¹⁴. This presents a significant problem because these are the same groups who are most severely affected by COVID-19.

As SARS-CoV-2 enters through the mucosal system, a vaccine that elicits mucosal antibodies at its point of entry may be advantageous. Furthermore, SARS-CoV-2 binds to the ACE2 receptors that are in many different organs and are abundant in respiratory and gastrointestinal tissues. Therefore, a vaccine that elicits both a systemic and a mucosal response may be beneficial. Injected vaccines generally elicit a poor mucosal but a strong systemic response while mucosal vaccines generally provide a strong mucosal but poor systemic response. It is unclear at this time what type of vaccine will offer the greatest protection from COVID-19.

Having medical personnel available to inject large populations is a logistical challenge, particularly in areas where skilled medical personnel is limiting. There is also the problem of compliance. Booster doses may be required for the vaccine to be fully effective, and individuals can be reluctant to schedule these. To illustrate this problem, 35% of first responders in industrial countries do not get the recommended boosters for hepatitis B despite the fact that they are the most vulnerable and are well aware of the risk.

Most vaccines are not heat stable and therefore shipping and storage require the cold chain. This adds cost and presents a major problem for COVID-19 where the cold chain is unreliable in many parts of the world.

A COVID-19 vaccine is needed that addresses the many problems facing mass immunization including: 1) efficacy for the general population as well as for poor responders, 2) easy to deliver, 3) rapidly scaled, 4) heat stable and 5) low cost. Based on our experience with other vaccines, we can predict that it is highly unlikely that the first generation of COVID-19 vaccines will meet these criteria. Also, as there have been three serious outbreaks of coronaviruses in the last 17 years (SARS, MERS and COVID-19), the probability is high that we will see another coronavirus outbreak in the next 5-10 years.

### SUMMARY

A vaccine for SARS CoV-2 vaccine is provided which is produced from a plant. A construct is introduced into a plant comprising a promoter preferentially directing expression to seed of the plant, a nucleic acid molecule encoding a Spike polypeptide of SARS-CoV-2 and a nucleic acid molecule targeting expression to the endoplasmic reticulum. Embodiments provide the construct and plasmids for expression of the same at high levels in plants. Expression levels of at least 10 mg/kg of seed of the plant are obtained. When the plant or plant product is orally administered to an animal, (including human), a protective response is observed

### BRIEF DESCRIPTION OF THE DRAWINGS

FIGS. 1A and 1B. hACE2 mice were administered SARS-CoV-2 and symptoms monitored. Body weight (FIG. 1A) and viral load in different tissues (FIG. 1B) were monitored. Results show the virus could infect the mice and cause clinical symptoms.

FIG. 2. Experimental design: SARS-Cov-2 challenge of immunized macaques.

FIG. 3. SARS-CoV S protein diagram from Jiang, et al. (2020) Emerging Microbes & Infections. 9: 275-277.

FIG. 4. COA pr44-BAASS:Cov-S1 and sequence. Legend: Underlined: key restriction enzyme sites intentionally included (NcoI, PacI); Underlined italic: Initiating methionine; italics/gray: signal sequence or added carrier peptide sequence (BAASS, KDEL, LTB, DC-peptide); bold italic: added valine/glycine for N-end stability; Stop codon/bold underlined

FIG. 5. COB pr44-BAASS:Cov-S1 KDEL Legend: Underlined: key restriction enzyme sites intentionally included (NcoI, PacI); Underlined italic: Initiating methionine; italics: signal sequence or added carrier peptide sequence (BAASS, KDEL, LTB, DC-peptide); bold italic/gray: added valine/glycine for N-end stability; Stop codon/bold underlined

FIG. 6. COC pr44-BAASS:Cov-S1-DC peptide Legend: Underlined: key restriction enzyme sites intentionally included (NcoI, PacI); Underlined italic: Initiating methionine; italics: signal sequence or added carrier peptide sequence (BAASS, KDEL, LTB, DC-peptide); bold italic/gray: added valine/glycine for N-end stability; Stop codon/bold underlined

FIG. 7. COD pr44-BAASS:Cov-S1 LTB Legend: Underlined: key restriction enzyme sites intentionally included (NcoI, PacI); Underlined italic: Initiating methionine; italics/gray: signal sequence or added carrier peptide sequence (BAASS, KDEL, LTB, DC-peptide); bold italic: added valine/glycine for N-end stability; Stop codon/bold underlined

FIG. 8. Western Blot of seed grain expression of SARS-CoV-2 (2019-nCoV) Spike Protein (S1 Subunit). The sequence for the S protein including the S1 domain was used to transform maize. Seeds were harvested and analyzed by Western blots to detect for S protein. Lane 2 is the S protein obtained from a commercial supplier. Lanes 3-10 are individual seeds from first generation plants expected to segregate 50% for the gene. Lanes 3, 5 and 10 show specific bands at the expected MW of ˜120 kD.

FIGS. 9A, 9B, 9C, and 9D. Response in young male and female (equal sex distribution) hACE2 mice after oral administration of maize-produced S protein were challenged with a Delta strain of SARS-CoV-2. Mice were given 2 oral doses of maize-produced S antigen and then challenged with 10,000 plaque forming units of a Delta strain of SARS-CoV-2. The mice were then monitored for 14 days to collect the flowing data; a) mean weight of mice that survived the challenge (FIG. 9A), b) survival rate (FIG. 9B), c) mean of antibody titer of individual mice (FIG. 9C) and d) neutralization activity (FIG. 9D).

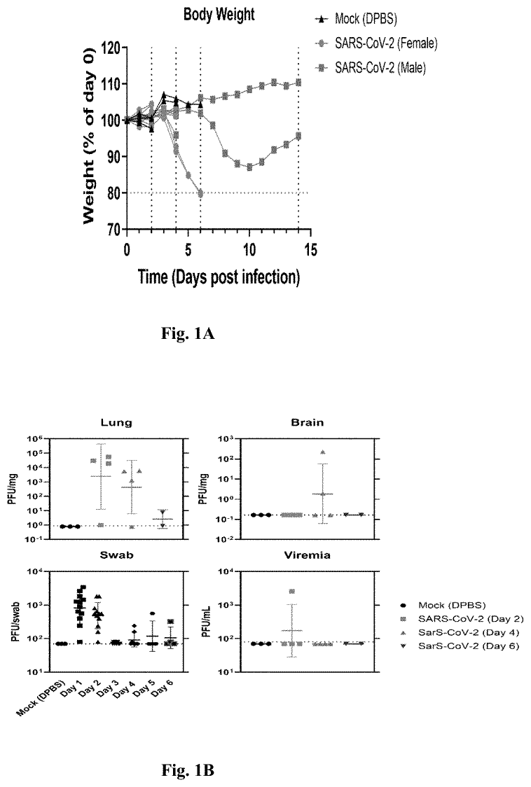

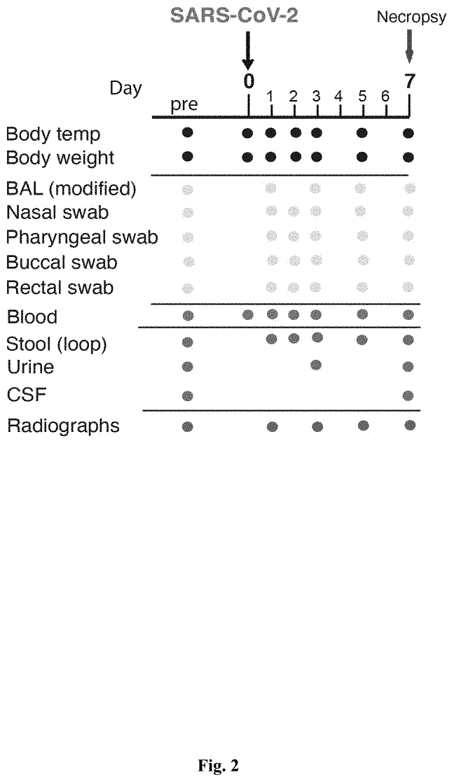

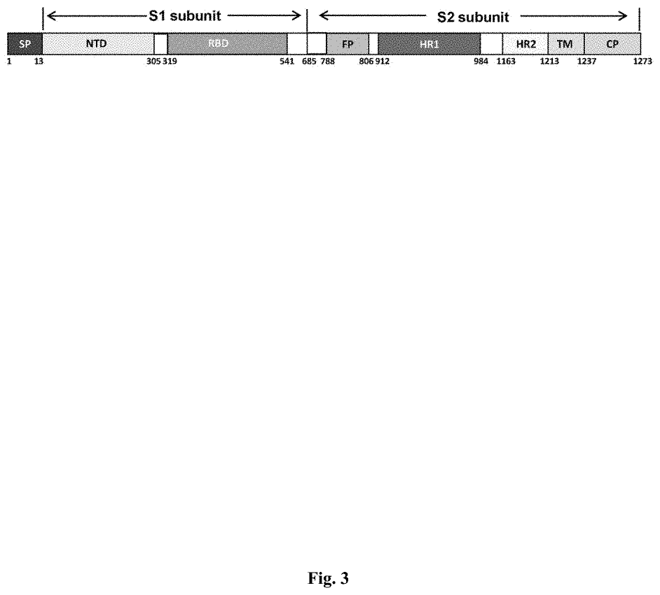

### DESCRIPTION

The S protein is expressed poorly in recombinant systems; therefore, it is difficult to develop a commercial subunit vaccine. Here in an embodiment, maize grain is used as a basis for the production of the subunit vaccine. High expression levels of at least 10 mg/kg of whole seed are obtained. An embodiment provides for a range of about 10-100 mg/kb. Further embodiments provide for expression at 11 mg/kb, 12 mg/kg, 13, 14, 15, 16, 17, 18, 19, 20, 25, 30, 35, 40 mg/kg of whole seed or more or amounts in-between.

Further, oral administration of the plant, plant part or a product produced from the plant part, such as a seed, grain, flour or other edible composition comprising the plant, plant part or product produced therefrom comprising the Spike protein results in protection against challenge for the subject animal. The subject animal may or may not produce antibodies in response, but the animal will have decreased morbidity or mortality resulting from administration of the vaccine, such that upon exposure to disease challenge, the animal is able to combat the infection. The compositions of the invention may also induce a serum response as well as a mucosal response. The serum response in an embodiment is within the range of two to 100-fold more than the control. In another embodiment the response can be 5 times, 10 times, 15 times, 20 times, 25 times, 30 times, 35 times, 40 times, 45 times, 50 times, 55 times, 60 times, 65 times, 70 times, 75 times, 80 times, 85 times, 90 times, 95 times or more greater than control animals not receiving vaccination, or amounts in-between.

As used herein, the term “animal” or “subject” or “subject animal” is intended to include human beings.

As used herein, the terms nucleic acid or polynucleotide refer to deoxyribonucleotides or ribonucleotides and polymers thereof in either single- or double-stranded form. The sequence used to make the vaccine may be obtained from any source, such as a biological source in isolating from a biological sample or can refer to a sequence synthetically produced based upon the sequence obtained from the sample. As such, the terms include RNA and DNA, which can be a gene or a portion thereof, a cDNA, a synthetic polydeoxyribonucleic acid sequence, or the like, and can be single-stranded or double-stranded, as well as a DNA/RNA hybrid. Furthermore, the terms are used herein to include naturally-occurring nucleic acid molecules, which can be isolated from a cell, as well as synthetic molecules, which can be prepared, for example, by methods of chemical synthesis or by enzymatic methods such as by the polymerase chain reaction (PCR). Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene.

Nucleic acids employed here include those that encode an entire polypeptide as well as those that encode a subsequence of the polypeptide or produce a fragment that provides a protective response. For example, nucleic acids that encode a polypeptide which is not full-length but nonetheless has protective activity against SARS-COV-2. The invention includes not only nucleic acids that include the nucleotide sequences as set forth herein, but also nucleic acids that are substantially identical to, correspond to, or substantially complementary to, the exemplified embodiments. For example, the invention includes nucleic acids that include a nucleotide sequence that is at least about 70% identical to one that is set forth herein, more preferably at least 75%, still more preferably at least 80%, more preferably at least 85%, 85.5% 86%, 86.5% 87%, 87.5% 88%, 88.5%, 89%, 89.5% still more preferably at least 90%, 90.5%, 91%, 91.5% 92%, 92.5%, 93%,94.5%, 94%, 94.5% and even more preferably at least about 95%, 95.5%, 96%, 96.5%, 97%, 97.5%, 98%, 98.5%, 99%, 95.5%, 100% identical (or any percentage in between) to an exemplified nucleotide sequence. The nucleotide sequence may be modified as described previously, so long as any polypeptide encoded produced is capable of inducing the generation of a protective response.

The nucleic acids can be obtained using methods that are known to those of skill in the art. Suitable nucleic acids (e.g., cDNA, genomic, or subsequences) can be cloned, or amplified by in vitro methods such as the polymerase chain reaction (PCR) using suitable primers, the ligase chain reaction (LCR), the transcription-based amplification system (TAS), or the self-sustained sequence replication system (SSR). A wide variety of cloning and in vitro amplification methodologies are well-known to persons of skill Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (2001) Molecular Cloning—A Laboratory Manual (Third ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864. Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Amheim & Levinson (Oct. 1, 1990) C& EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039. Nucleic acids or subsequences of these nucleic acids, can be prepared by any suitable method as described above, including, for example, cloning and restriction of appropriate sequences.

“Codon optimization” can be used to optimize sequences for expression in an animal and is defined as modifying a nucleic acid sequence for enhanced expression in the cells of the animal of interest, e.g. swine, by replacing at least one, more than one, or a significant number, of codons of the native sequence with codons that are more frequently or most frequently used in the genes of that animal. Various species exhibit particular bias for certain codons of a particular amino acid.

As used herein, a “polypeptide” refers generally to peptides and proteins. In certain embodiments the polypeptide may be at least two, three, four, five, six, seven, eight, nine or ten or more amino acids or more or any amount in-between. A peptide is generally considered to be more than fifty amino acids. The terms “fragment,” “derivative” and “homologue” when referring to the polypeptides according to the present invention, means a polypeptide which retains essentially the same biological function or activity as said polypeptide, that is, act as an antigen and/or provide treatment for and/or protection against disease. Such fragments, derivatives and homologues can be chosen based on the ability to retain one or more of the biological activities of the polypeptide, that is, act as an antigen and/or provide treatment for and/or protection against the pathogen. The polypeptide vaccines of the present invention may be recombinant polypeptides, natural polypeptides or synthetic polypeptides, preferably recombinant polypeptides. One skilled in the art appreciates that it is possible that the protective polypeptide may be expressed by the gene in the host cells and the plant composition administered to the animal or extracted from the plant prior to administration.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode a protective polypeptide are preferably optimized for expression in a particular host cell (e.g., yeast, mammalian, plant, fungal, and the like) used to produce the polypeptide or RNA.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” referred to herein as a “variant” where the alteration results in the substitution of an amino acid with a chemically similar amino acid. Conservative substitution tables providing functionally similar amino acids are well known in the art. See, for example, Davis et al., “Basic Methods in Molecular Biology” Appleton & Lange, Norwalk, Conn. (1994). Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

The isolated variant proteins can be purified from cells that naturally express it, purified from cells that have been altered to express it (recombinant), or synthesized using known protein synthesis methods. For example, a nucleic acid molecule encoding the variant polypeptide is cloned into an expression vector, the expression vector introduced into a host cell and the variant protein expressed in the host cell. The variant protein can then be isolated from the cells by an appropriate purification scheme using standard protein purification techniques. Many of these techniques are described in detail below.

The methods include amino acids that include an amino acid sequence that is at least about 70% identical to one that is set forth herein, more preferably at least 75%, still more preferably at least 80%, more preferably at least 85%, 85.5% 86%, 86.5% 87%, 87.5% 88%, 88.5%, 89%, 89.5% still more preferably at least 90%, 90.5%, 91%, 91.5% 92%, 92.5%, 93%,94.5%, 94%, 94.5% and even more preferably at least about 95%, 95.5%, 96%, 96.5%, 97%, 97.5%, 98%, 98.5%, 99%, 95.5%, 100% identical (or any percentage in between) to an exemplified nucleotide sequence. The sequence may be modified as described previously, so long the polypeptide is capable of inducing the generation of a protective response.

The variant proteins used in the present methods can be attached to heterologous sequences to form chimeric or fusion proteins. Such chimeric and fusion proteins comprise a variant protein fused in-frame to a heterologous protein having an amino acid sequence not substantially homologous to the variant protein. The heterologous protein can be fused to the N-terminus or C-terminus of the variant protein.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., Current Protocols in Molecular Biology, 1992). Moreover, many expression vectors are commercially available that already encode a fusion moiety (e.g., a GST protein). A variant protein-encoding nucleic acid can be cloned into such an expression vector such that the fusion moiety is linked in-frame to the variant protein.

Polypeptides sometimes contain amino acids other than the 20 amino acids commonly referred to as the 20 naturally occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art. Accordingly, the variant peptides of the present invention also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The present methods further provide functional fragments of the nucleic acid molecules and polypeptides including variant proteins of the polyeptide, in addition to proteins and peptides that comprise and consist of such fragments, provided that such fragments act as an antigen and/or provide treatment for and/or protection against SARS-COV-2.

As used herein, the term “subunit” refers to a portion of the microorganism which provides protection and may itself be antigenic, i.e., capable of inducing an immune response in an animal. The term should be construed to include subunits which are obtained by both recombinant and biochemical methods.

In one embodiment, a method of identifying protective sequences of the virus or nucleic acids that elicit protection is provided. This method also includes fragments, derivatives, or homologs of the nucleic acid molecule. In one aspect, the method comprises administering to a test animal such sequences. The test and control animals are subsequently challenged with an infectious amount of a microorganism that causes the disease. Various methods and techniques for determining whether protection is provided are known to those skilled in the art, including but not limited to, observing a difference between the test and control animal in the symptoms of the disease, for example. A decrease in any of the symptoms observed in the test animal compared to the control animal indicates that protective molecule(s) provide a degree of protection against disease. Similar symptoms or an increase in any of the symptoms observed in the test animal compared to those observed in the control animal indicate that the protective molecule(s) do not provide protection.

In another aspect, determining whether the molecules provided protection against SARS-CoV-2 includes determining the presence or absence of challenge disease in the test animal by electron microscopy or antibody or assays such as the fluorescent focusing neutralizing (FFN) test or Western blot assay may be used. PCR methods may be used to determine if the protective molecule is present. Northern blotting can detect the presence of diagnostic sequences. In another aspect, an ELISA or similar assay, such as a hemagglutinin inhibition assay are the types of many varied assays that can determine if the protective molecule is effective. The ELISA or enzyme linked immunoassay has been known since 1971. In general, antigens solubilized in a buffer are coated on a plastic surface. When serum is added, antibodies can attach to the antigen on the solid phase. The presence or absence of these antibodies can be demonstrated when conjugated to an enzyme. Adding the appropriate substrate will detect the amount of bound conjugate which can be quantified. A common ELISA assay is one which uses biotinylated anti-(protein) polyclonal antibodies and an alkaline phosphatase conjugate. For example, an ELISA used for quantitative determination of protein levels can be an antibody sandwich assay, which utilizes polyclonal rabbit antibodies obtained commercially. The antibody is conjugated to alkaline phosphatases for detection. In another example, an ELISA assay to detect trypsin or trypsinogen uses biotinylated anti-trypsin or anti-trypsinogen polyclonal antibodies and a streptavidin-alkaline phosphatase conjugate.

Clearly, many such methods are available to one skilled in the art to ascertain if the molecule provides protection and provides protection at the levels administered to the animal.

The nucleic acid molecule, polypeptide or fragment thereof, when administered to the subject animal produces a protective response to SARS-COV-2. A protective response is elicited in the animal. The subject animal may or may not produce antibodies in response, but the animal will have decreased morbidity or mortality resulting from administration of the vaccine, and as described further herein. The terms “protecting”, “protection”, “protective immunity” or “protective immune response,” as used herein, are intended to mean that the host subject animal mounts an active immune response to the vaccine or polypeptides of the present invention, such that upon exposure to disease challenge, the subject animal is able to combat the infection. Thus, a protective immune response will decrease the incidence of morbidity and mortality from exposure to the microorganism among a host animal. The subject animal will be protected from subsequent exposure to the disease-causing agent. In an embodiment, the animal may be protected by treating the animal which has already been exposed to the disease-causing agent by administration of the vaccine or polypeptide after such exposure. In such an instance there is also shown to be a lessening of morbidity and mortality. Those skilled in the art will understand that in a commercial animal setting, the production of a protective immune response may be assessed by evaluating the effects of vaccination on the herd as a whole, e.g., there may still be morbidity and mortality in a minority of vaccinated animals. Furthermore, protection also includes a lessening in severity of any gross or histopathological changes and/or of symptoms of the disease, as compared to those changes or symptoms typically caused by the isolate in similar animals which are unprotected (i.e., relative to an appropriate control). Thus, a protective immune response will decrease the symptoms of the disease compared to a control animal.

A “construct” is a package of genetic material inserted into the genome of a cell via various techniques. A “vector” is any means for the transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA or RNA replication in vivo, i.e., capable of replication under its own control. The term “vector” includes both viral and nonviral means for introducing the nucleic acid into a cell in vitro, ex vivo or in vivo. Viral vectors include alphavirus, retrovirus, adeno-associated virus, pox, baculovirus, vaccinia, herpes simplex, Epstein-Barr, rabies virus, vesicular stomatitis virus, and adenovirus vectors. Non-viral vectors include, but are not limited to plasmids, liposomes, electrically charged lipids (cytofectins), DNA- or RNA protein complexes, and biopolymers. In addition to a nucleic acid, a vector may also contain one or more regulatory regions, and/or selectable markers useful in selecting, measuring, and monitoring nucleic acid transfer results (transfer to which tissues, duration of expression, etc.).

A “cassette” refers to a segment of DNA that can be inserted into a vector at specific restriction sites. The segment of DNA encodes a polypeptide of interest or produces RNA, and the cassette and restriction sites are designed to ensure insertion of the cassette in the proper reading frame for transcription and translation.

A nucleic acid molecule is introduced into a cell when it is inserted in the cell. A cell has been “transfected” by exogenous or heterologous DNA or RNA when such DNA or RNA has been introduced inside the cell.

A cell has been “transformed” by exogenous or heterologous DNA or RNA when the transfected DNA or RNA effects a phenotypic change. The transforming DNA can be integrated (covalently linked) into chromosomal DNA making up the genome of the cell.

Once the gene is engineered to contain desired features, such as the desired subcellular localization sequences, it may then be placed into an expression vector by standard methods. The selection of an appropriate expression vector will depend upon the method of introducing the expression vector into host cells. A typical expression vector contains prokaryotic DNA elements coding for a bacterial origin of replication and an antibiotic resistance gene to provide for the growth and selection of the expression vector in the bacterial host; a cloning site for insertion of an exogenous DNA sequence; eukaryotic DNA elements that control initiation of transcription of the exogenous gene; and DNA elements that control the processing of transcripts, such as transcription termination/polyadenylation sequences. It also can contain such sequences as are needed for the eventual integration of the vector into the host chromosome.

By “promoter” is meant a regulatory region of DNA capable of regulating the transcription of a sequence linked thereto. It usually comprises a TATA box capable of directing RNA polymerase II to initiate RNA synthesis at the appropriate transcription initiation site for a particular coding sequence. The promoter is the minimal sequence sufficient to direct transcription in a desired manner. The term “regulatory region” is also used to refer to the sequence capable of initiating transcription in a desired manner.

A nucleic acid molecule may be used in conjunction with its own or another promoter. In one embodiment, a selection marker a nucleic acid molecule of interest can be functionally linked to the same promoter. In another embodiment, they can be functionally linked to different promoters. In yet third and fourth embodiments, the expression vector can contain two or more genes of interest that can be linked to the same promoter or different promoters. For example, one promoter can be used to drive a nucleic acid molecule of interest and the selectable marker, or a different promoter used for one or each. These other promoter elements can be those that are constitutive or sufficient to render promoter-dependent gene expression controllable as being cell-type specific, tissue-specific or time or developmental stage specific, or being inducible by external signals or agents. Such elements may be located in the 5′ or 3′ regions of the gene. Although the additional promoter may be the endogenous promoter of a structural gene of interest, the promoter can also be a foreign regulatory sequence. Promoter elements employed to control expression of product proteins and the selection gene can be any host-compatible promoters. These can be plant gene promoters, such as, for example, the ubiquitin promoter (European patent application no. 0 342 926); the promoter for the small subunit of ribulose-1,5-bis-phosphate carboxylase (ssRUBISCO) (Coruzzi et al., 1984; Broglie et al., 1984); or promoters from the tumor-inducing plasmids from Agrobacterium tumefaciens, such as the nopaline synthase, octopine synthase and mannopine synthase promoters (Velten and Schell, 1985) that have plant activity; or viral promoters such as the cauliflower mosaic virus (CaMV) 19S and 35S promoters (Guilley et al., 1982; Odell et al., 1985), the figwort mosaic virus FLt promoter (Maiti et al., 1997) or the coat protein promoter of TMV (Grdzelishvili et al., 2000). Alternatively, plant promoters such as heat shock promoters for example soybean hsp 17.5-E (Gurley et al., 1986); or ethanol-inducible promoters (Caddick et al., 1998) may be used. See International Patent Application No. WO 91/19806 for a review of illustrative plant promoters suitably employed.

A promoter can additionally comprise other recognition sequences generally positioned upstream or 5′ to the TATA box, referred to as upstream promoter elements, which influence the transcription initiation rate. It is recognized that having identified the nucleotide sequences for a promoter region, it is within the state of the art to isolate and identify further regulatory elements in the 5′ region upstream from the particular promoter region identified herein. Thus, the promoter region is generally further defined by comprising upstream regulatory elements such as those responsible for tissue and temporal expression of the coding sequence, enhancers and the like.

Tissue-preferred promoters can be utilized to target enhanced transcription and/or expression within a particular tissue. When referring to preferential expression, what is meant is expression at a higher level in the particular tissue than in other tissue. Examples of these types of promoters include seed preferred expression such as that provided by the phaseolin promoter (Bustos et al. (1989) The Plant Cell Vol. 1, 839-853). For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa zein, γ-zein, waxy, shrunken 1, shrunken 2, an Ltp1 (See, for example, U.S. Pat. No. 7,550,579), an Ltp2 (Opsahl-Sorteberg, H-G. et al., (2004) Gene 341:49-58 and U.S. Pat. No. 5,525,716), and oleosin genes. See also WO 00/12733, where seed-preferred promoters from end1 and end2 genes are disclosed. Seed-preferred promoters also include those promoters that direct gene expression predominantly to specific tissues within the seed such as, for example, the endosperm-preferred promoter of γ-zein, the cryptic promoter from tobacco (Fobert et al. (1994) “T-DNA tagging of a seed coat-specific cryptic promoter in tobacco” Plant J. 4: 567-577), the P-gene promoter from corn (Chopra et al. (1996) “Alleles of the maize P gene with distinct tissue specificities encode Myb-homologous proteins with C-terminal replacements” Plant Cell 7:1149-1158, Erratum in Plant Cell 1997, 1:109), the globulin-1 promoter from corn (Belanger and Kriz (1991) “Molecular basis for Allelic Polymorphism of the maize Globulin-1 gene” Genetics 129: 863-972 and GenBank accession No. L22344), promoters that direct expression to the seed coat or hull of corn kernels, for example the pericarp-specific glutamine synthetase promoter (Muhitch et al., (2002) “Isolation of a Promoter Sequence From the Glutamine Synthetase₁₋₂ Gene Capable of Conferring Tissue-Specific Gene Expression in Transgenic Maize” Plant Science 163:865-872 and GenBank accession number AF359511) and to the embryo (germ) such as that disclosed at U.S. Pat. No. 7,169,967. When referring to a seed or an embryo preferred promoter is meant that it expresses an operably linked sequence to a higher degree in seed or embryo tissue that in other plant tissue. It may express during seed or embryo development, along with expression at other stages, may express strongly during seed or embryo development and to a much lesser degree at other times.

The range of available promoters includes inducible promoters. An inducible regulatory element is one that is capable of directly or indirectly activating transcription of one or more DNA sequences or genes in response to an inducer. In the absence of an inducer the DNA sequences or genes will not be transcribed. Typically, the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. Typically, the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the actin of a pathogen or disease agent such as a virus. A cell containing an inducible regulatory element may be exposed to an inducer by externally applying the inducer to the cell or plant such as by spraying, watering, heating or similar methods.

Any inducible promoter can be used. See Ward et al. Plant Mol. Biol. 22: 361-366 (1993). Exemplary inducible promoters include ecdysone receptor promoters, U.S. Pat. No. 6,504,082; promoters from the ACE1 system which responds to copper (Mett et al. PNAS 90: 4567-4571 (1993)); In2-1 and In2-2 gene from maize which respond to benzenesulfonamide herbicide safeners (U.S. Pat. No. 5,364,780; Hershey et al., Mol. Gen. Genetics 227: 229-237 (1991) and Gatz et al., Mol. Gen. Genetics 243: 32-38 (1994)) Tet repressor from Tn10 (Gatz et al., Mol. Gen. Genet. 227: 229-237 (1991); or from a steroid hormone gene, the transcriptional activity of which is induced by a glucocorticosteroid hormone. Schena et al., Proc. Natl. Acad. Sci. U.S.A. 88: 10421 (1991); the maize GST promoter, which is activated by hydrophobic electrophilic compounds that are used as pre-emergent herbicides; and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156).

Other components of the vector may be included, also depending upon intended use of the gene. Examples include selectable markers, targeting or regulatory sequences, stabilizing or leader sequences, introns etc. General descriptions and examples of plant expression vectors and reporter genes can be found in Gruber, et al., “Vectors for Plant Transformation” in Method in Plant Molecular Biology and Biotechnology, Glick et al eds; CRC Press pp. 89-119 (1993). The selection of an appropriate expression vector will depend upon the host and the method of introducing the expression vector into the host. The expression cassette will also include at the 3′ terminus of the heterologous nucleotide sequence of interest, a transcriptional and translational termination region functional in plants.

The expression vector can optionally also contain a signal sequence located between the promoter and the gene of interest and/or after the gene of interest. A signal sequence is a nucleotide sequence, translated to give an amino acid sequence, which is used by a cell to direct the protein or polypeptide of interest to be placed in a particular place within or outside the eukaryotic cell. Many signal sequences are known in the art. See, for example Becker et al., (1992) Plant Mol. Biol. 20:49, Knox, C., et al., “Structure and Organization of Two Divergent Alpha-Amylase Genes from Barley”, Plant Mol. Biol. 9:3-17 (1987), Lerner et al., (1989) Plant Physiol. 91:124-129, Fontes et al., (1991) Plant Cell 3:483-496, Matsuoka et al., (1991) Proc. Natl. Acad. Sci. 88:834, Gould et al., (1989) J. Cell. Biol. 108:1657, Creissen et al., (1991) Plant J. 2:129, Kalderon, et al., (1984) “A short amino acid sequence able to specify nuclear location,” Cell 39:499-509, Steifel, et al., (1990) “Expression of a maize cell wall hydroxyproline-rich glycoprotein gene in early leaf and root vascular differentiation” Plant Cell 2:785-793. When targeting the protein to the cell wall use of a signal sequence is necessary. One example is the barley alpha-amylase signal sequence. Rogers, J. C. (1985) “Two barley alpha-amylase gene families are regulated differently in aleurone cells” J. Biol. Chem. 260: 3731-3738.

In those instances where it is desirable to have the expressed product of the heterologous nucleotide sequence directed to a particular organelle, particularly the plastid, amyloplast, or to the endoplasmic reticulum, or secreted at the cell's surface or extracellularly, the expression cassette can further comprise a coding sequence for a transit peptide. Such transit peptides are well known in the art and include, but are not limited to, the transit peptide for the acyl carrier protein, the small subunit of RUBISCO, plant EPSP synthase, Zea mays Brittle-1 chloroplast transit peptide (Nelson et al. Plant Physiol 117(4):1235-1252 (1998); Sullivan et al. Plant Cell 3(12):1337-48; Sullivan et al., Planta (1995) 196(3):477-84; Sullivan et al., J. Biol. Chem. (1992) 267(26):18999-9004) and the like. One skilled in the art will readily appreciate the many options available in expressing a product to a particular organelle. Use of transit peptides is well known (e.g., see U.S. Pat. Nos. 5,717,084; 5,728,925). A protein may be targeted to the endoplasmic reticulum of the plant cell. This may be accomplished by use of a localization sequence, such as KDEL. This sequence (Lys-Asp-Glu-Leu) contains the binding site for a receptor in the endoplasmic reticulum. (Munro et al., (1987) “A C-terminal signal prevents secretion of luminal ER proteins.” Cell. 48:899-907. There are a wide variety of endoplasmic reticulum retention signal sequences available to one skilled in the art and the KDEL sequence is one example. Another example is HDEL (His-Asp-Glu-Leu (SEQ ID NO: 24)). See, for example, Kumar et al. which discuses methods of producing a variety of endoplasmic reticulum proteins. Kumar et al. (2017) “prediction of endoplasmic reticulum resident proteins using fragmented amino acid composition and support vector machine” Peer J. doi: 10.7717/peerj.3561.

Retaining the protein in the vacuole is another example. Signal sequences to accomplish this are well known. For example, Raikhel U.S. Pat. No. 5,360,726 shows a vacuole signal sequence as does Warren et al at U.S. Pat. No. 5,889,174. Vacuolar targeting signals may be present either at the amino-terminal portion, (Holwerda et al., (1992) The Plant Cell, 4:307-318, Nakamura et al., (1993) Plant Physiol., 101:1-5), carboxy-terminal portion, or in the internal sequence of the targeted protein. (Tague et al., (1992) The Plant Cell, 4:307-318, Saalbach et al. (1991) The Plant Cell, 3:695-708). Additionally, amino-terminal sequences in conjunction with carboxy-terminal sequences are responsible for vacuolar targeting of gene products (Shinshi et al. (1990) Plant Molec. Biol. 14:357-368).

The termination region can be native with the promoter nucleotide sequence can be native with the DNA sequence of interest or can be derived from another source. Convenient termination regions are available from the Ti-plasmid of A, tumefaciens, such as the octopine synthase (MacDonald et al., (1991) Nuc. Acids Res. 19(20)5575-5581) and nopaline synthase termination regions (Depicker et al., (1982) Mol. and Appl. Genet. 1:561-573 and Shaw et al. (1984) Nucleic Acids Research Vol. 12, No. 20 pp7831-7846 (nos). Examples of various other terminators include the pin II terminator from the protease inhibitor II gene from potato (An, et al. (1989) Plant Cell 1, 115-122. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Many variations on the promoters, selectable markers, signal sequences, leader sequences, termination sequences, introns, enhancers and other components of the vector are available to one skilled in the art.

The term plant refers to the entire plant or plant material or plant part or plant tissue or plant cell including a collection of plant cells. It is used broadly herein to include any plant at any stage of development, or to part of a plant, including a plant cutting, a plant cell culture, a plant organ, a plant seed, and a plantlet. Plant seed parts, for example, include the pericarp or kernel, the embryo or germ, and the endoplasm. A plant cell is the structural and physiological unit of the plant, comprising a protoplast and a cell wall. A plant cell can be in the form of an isolated single cell or aggregate of cells such as a friable callus, or a cultured cell, or can be part of a higher organized unit, for example, a plant tissue, plant organ, or plant. Thus, a plant cell can be a protoplast, a gamete producing cell, or a cell or collection of cells that can regenerate into a whole plant. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like. In an embodiment, the tissue culture will preferably be capable of regenerating plants. Preferably, the regenerable cells in such tissue cultures will be embryos, protoplasts, meristematic cells, callus, pollen, leaves, anthers, roots, root tips, silk, flowers, kernels, ears, cobs, husks or stalks. Still further, plants may be regenerated from the tissue cultures.

Any plant species may be used, whether monocotyledonous or dicotyledonous, including but not limited to corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), oats (Avena), barley (Hordeum), vegetables, ornamentals, and conifers. Vegetables include tomatoes (Lycopersicon esculentum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers which may be employed in practicing the present invention include, for example, pines such as loblolly pine (Pinus taeda), slash pine (Pinus elliotii), ponderosa pine (Pinus ponderosa), lodgepole pine (Pinus contotta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis) algae, or Lemnoideae (aka Duckweed). An embodiment provides the plant is maize.

The method of transformation/transfection is not critical; various methods of transformation or transfection are currently available. As newer methods are available to transform crops or other host cells they may be directly applied. Accordingly, a wide variety of methods have been developed to insert a DNA sequence into the genome of a host cell to obtain the transcription or transcript and translation of the sequence to effect phenotypic changes in the organism. Thus, any method which provides for efficient transformation/transfection may be employed.

Methods for introducing expression vectors into plant tissue available to one skilled in the art are varied and will depend on the plant selected. Procedures for transforming a wide variety of plant species are well known and described throughout the literature. (See, for example, Miki and McHugh (2004) Biotechnol. 107, 193-232; Klein et al. (1992) Biotechnology (N Y) 10, 286-291; and Weising et al. (1988) Annu. Rev. Genet. 22, 421-477). For example, the DNA construct may be introduced into the genomic DNA of the plant cell using techniques such as microprojectile-mediated delivery (Klein et al. 1992, supra), electroporation (Fromm et al., 1985 Proc. Natl. Acad. Sci. USA 82, 5824-5828), polyethylene glycol (PEG) precipitation (Mathur and Koncz, 1998 Methods Mol. Biol. 82, 267-276), direct gene transfer (WO 85/01856 and EP-A-275 069), in vitro protoplast transformation (U.S. Pat. No. 4,684,611), and microinjection of plant cell protoplasts or embryogenic callus (Crossway, A. (1985) Mol. Gen. Genet. 202, 179-185). Agrobacterium transformation methods of Ishida et al. (1996) and also described in U.S. Pat. No. 5,591,616 are yet another option. Co-cultivation of plant tissue with Agrobacterium tumefaciens is a variation, where the DNA constructs are placed into a binary vector system (Ishida et al., 1996 Nat. Biotechnol. 14, 745-750). The virulence functions of the Agrobacterium tumefaciens host will direct the insertion of the construct into the plant cell DNA when the cell is infected by the bacteria. See, for example, Fraley et al. (1983) Proc. Natl. Acad. Sci. USA, 80, 4803-4807. Agrobacterium is primarily used in dicots, but monocots including maize can be transformed by Agrobacterium. See, for example, U.S. Pat. No. 5,550,318. In one of many variations on the method, Agrobacterium infection of corn can be used with heat shocking of immature embryos (Wilson et al. U.S. Pat. No. 6,420,630) or with antibiotic selection of Type II callus (Wilson et al., U.S. Pat. No. 6,919,494).

Rice transformation is described by Hiei et al. (1994) Plant J. 6, 271-282 and Lee et al. (1991) Proc. Nat. Acad. Sci. USA 88, 6389-6393. Standard methods for transformation of canola are described by Moloney et al. (1989) Plant Cell Reports 8, 238-242. Corn transformation is described by Fromm et al. (1990) Biotechnology (N Y) 8, 833-839 and Gordon-Kamm et al. (1990) supra. Wheat can be transformed by techniques similar to those used for transforming corn or rice. Sorghum transformation is described by Casas et al. (Casas et al. (1993). Transgenic sorghum plants via microprojectile bombardment. Proc. Natl. Acad. Sci. USA 90, 11212-11216) and barley transformation is described by Wan and Lemaux (Wan and Lemaux (1994) Generation of large numbers of independently transformed fertile barley plants. Plant Physiol. 104, 37-48). Soybean transformation is described in a number of publications, including U.S. Pat. No. 5,015,580.

In one method, the Agrobacterium transformation methods of Ishida et al. (1996) and also described in U.S. Pat. No. 5,591,616, are generally followed, with modifications that the inventors have found improve the number of transformants obtained. The Ishida method uses the A188 variety of maize that produces Type I callus in culture. In an embodiment the Hi II maize line is used which initiates Type II embryogenic callus in culture (Armstrong et al., 1991).

While Ishida recommends selection on phosphinothricin when using the bar or pat gene for selection, another preferred embodiment provides use of bialaphos instead. In general, as set forth in the U.S. Pat. No. 5,591,616 patent, and as outlined in more detail below, dedifferentiation is obtained by culturing an explant of the plant on a dedifferentiation-inducing medium for not less than seven days, and the tissue during or after dedifferentiation is contacted with Agrobacterium having the gene of interest. The cultured tissue can be callus, an adventitious embryo-like tissue or suspension cells, for example. In this preferred embodiment, the suspension of Agrobacterium has a cell population of 10⁶ to 10¹¹ cells/ml and are contacted for three to ten minutes with the tissue, or continuously cultured with Agrobacterium for not less than seven days. The Agrobacterium can contain plasmid pTOK162, with the gene of interest between border sequences of the T region of the plasmid, or the gene of interest may be present in another plasmid-containing Agrobacterium. The virulence region may originate from the virulence region of a Ti plasmid or Ri plasmid. The bacterial strain used in the Ishida protocol is LBA4404 with the 40 kb super binary plasmid containing three vir loci from the hypervirulent A281 strain. The plasmid has resistance to tetracycline. The cloning vector cointegrates with the super binary plasmid. Since the cloning vector has an E. coli specific replication origin, but not an Agrobacterium replication origin, it cannot survive in Agrobacterium without cointegrating with the super binary plasmid. Since the LBA4404 strain is not highly virulent, and has limited application without the super binary plasmid, the inventors have found in yet another embodiment that the EHA101 strain is preferred. It is a disarmed helper strain derived from the hypervirulent A281 strain. The cointegrated super binary/cloning vector from the LBA4404 parent is isolated and electroporated into EHA101, selecting for spectinomycin resistance. The plasmid is isolated to assure that the EHA101 contains the plasmid. EHA101 contains a disarmed pTi that carries resistance to kanamycin. See, Hood et al. (1986).

Further, the Ishida protocol as described provides for growing fresh culture of the Agrobacterium on plates, scraping the bacteria from the plates, and resuspending in the co-culture medium as stated in the U.S. Pat. No. 5,591,616 patent for incubation with the maize embryos. This medium includes 4.3 g MS salts, 0.5 mg nicotinic acid, 0.5 mg pyridoxine hydrochloride, 1.0 ml thiamine hydrochloride, casamino acids, 1.5 mg 2,4-D, 68.5 g sucrose and 36 g glucose per liter, all at a pH of 5.8. In a further preferred method, the bacteria are grown overnight in a 1 ml culture and then a fresh 10 ml culture is re-inoculated the next day when transformation is to occur. The bacteria grow into log phase and are harvested at a density of no more than OD₆₀₀=0.5, preferably between 0.2 and 0.5. The bacteria are then centrifuged to remove the media and resuspended in the co-culture medium. Since Hi II is used, medium preferred for Hi II is used. This medium is described in considerable detail by Armstrong and Green (1985). The resuspension medium is the same as that described above. All further Hi II media are as described in Armstrong and Green (1985). The result is redifferentiation of the plant cells and regeneration into a plant. Redifferentiation is sometimes referred to as dedifferentiation, but the former term more accurately describes the process where the cell begins with a form and identity, is placed on a medium in which it loses that identity and becomes “reprogrammed” to have a new identity. Thus, the scutellum cells become embryogenic callus.

A transgenic plant may be produced that contains an introduced nucleic acid molecule encoding the polypeptide.

When referring to introduction of a nucleotide sequence into a plant is meant to include transformation into the cell, as well as crossing a plant having the sequence with another plant, so that the second plant contains the heterologous sequence, as in conventional plant breeding techniques. Such breeding techniques are well known to one skilled in the art. This can be accomplished by any means known in the art for breeding plants such as, for example, cross pollination of the transgenic plants that are described above with other plants, and selection for plants from subsequent generations which express the amino acid sequence. The plant breeding methods used herein are well known to one skilled in the art. For a discussion of plant breeding techniques, see Poehlman (1995) Breeding Field Crops. AVI Publication Co., Westport Conn, 4^{th} Edit.). Many crop plants useful in this method are bred through techniques that take advantage of the plant's method of pollination. A plant is self-pollinating if pollen from one flower is transferred to the same or another flower of the same plant. A plant is cross-pollinating if the pollen comes from a flower on a different plant. For example, in Brassica, the plant is normally self-sterile and can only be cross-pollinated unless, through discovery of a mutant or through genetic intervention, self-compatibility is obtained. In self-pollinating species, such as rice, oats, wheat, barley, peas, beans, soybeans, tobacco and cotton, the male and female plants are anatomically juxtaposed. During natural pollination, the male reproductive organs of a given flower pollinate the female reproductive organs of the same flower. Maize plants (Zea mays L.) can be bred by both self-pollination and cross-pollination techniques. Maize has male flowers, located on the tassel, and female flowers, located on the ear, on the same plant. It can self or cross-pollinate.

Pollination can be by any means, including but not limited to hand, wind or insect pollination, or mechanical contact between the male fertile and male sterile plant. For production of hybrid seeds on a commercial scale in most plant species pollination by wind or by insects is preferred. Stricter control of the pollination process can be achieved by using a variety of methods to make one plant pool male sterile, and the other the male fertile pollen donor. This can be accomplished by hand detasseling, cytoplasmic male sterility, or control of male sterility through a variety of methods well known to the skilled breeder. Examples of more sophisticated male sterility systems include those described by Brar et al., U.S. Pat. Nos. 4,654,465 and 4,727,219 and Albertsen et al., U.S. Pat. Nos. 5,859,341 and 6,013,859.

Backcrossing methods may be used to introduce the gene into the plants. This technique has been used for decades to introduce traits into a plant. An example of a description of this and other plant breeding methodologies that are well known can be found in references such as Neal (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

Selection and propagation techniques described above can yield a plurality of transgenic plants that are harvested in a conventional manner. The plant or any parts expressing the recombinant polypeptide can be used in a commercial process, or the polypeptide extracted. When using the plant or part itself, it can, for example, be made into flour and then applied in the commercial process. Polypeptide extraction from biomass can be accomplished by known methods. Downstream processing for any production system refers to all unit operations after product synthesis, in this case protein production in transgenic seed (Kusnadi, A. R., Nikolov, Z. L., Howard, J. A., 1997. Biotechnology and Bioengineering. 56:473-484). For example, seed can be processed either as whole seed ground into flour or, fractionated and the germ separated from the hulls and endosperm. If germ is used, it is usually defatted using an extraction process and the remaining crushed germ ground into a meal or flour. In some cases, the germ is used directly in the process or the protein can be extracted (See, e.g., WO 98/39461). Extraction is generally made into aqueous buffers at specific pH to enhance recombinant protein extraction and minimize native seed protein extraction. Subsequent protein concentration or purification can follow.

The compositions and process described here are also to producing and administering a vaccine that protects an animal from SARS-COV-2.

As used herein, the term “vaccine” as used herein refers to a pharmaceutical composition comprising at least one protective molecule, that induces protective response in a subject and possibly, but not necessarily, one or more additional components that enhance the activity of said active component. A vaccine may additionally comprise further components typical to pharmaceutical compositions. In another form, the immunologically active component of a vaccine may comprise appropriate elements of said organisms (subunit vaccines) whereby these elements are generated either by destroying the whole organism or the growth cultures of such microorganisms and subsequent purification steps yielding in the desired structure(s), or by synthetic processes induced by an appropriate manipulation of a suitable system such as, but not restricted to, bacteria, insects, mammalian, or other species, plus subsequent isolation and purification procedures or by induction of said synthetic processes in the animal needing a vaccine by direct incorporation of genetic material using suitable pharmaceutical compositions (polynucleotide vaccination). A vaccine may comprise one or simultaneously more than one of the elements described above.

The present vaccines may include a pharmaceutically acceptable carrier, excipient, carrier, stabilizer and/or diluent. Without intending to be limiting, examples include wetting agents and lubricating agents, preservative agents, lipids, stabilizers, solubilizers and emulsifiers. The carrier can be a solvent or dispersion medium containing, for example, water, ethanol, polyol (for example, glycerol, propylene glycol, liquid polyethylene glycol, and the like), suitable mixtures thereof and vegetable oils. One possible carrier is a physiological salt solution. Examples of stabilizers include, for example, glycerol/EDTA, carbohydrates (such as sorbitol, mannitol, trehalose, starch, sucrose, dextran or glucose), proteins (such as albumin or casein) and protein degradation products (e.g., partially hydrolyzed gelatin).

It is possible to provide an adjuvant in the vaccine. Adjuvants enhance the immunogenicity of an antigen but are not necessarily immunogenic themselves. Adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses. Immunostimulatory agents or adjuvants have been used for many years to improve the host immune responses to, for example, vaccines. The vaccines of the present invention may be used in conjunction with an adjuvants, for example, lipopolysaccharides, aluminum hydroxide and aluminum phosphate (alum), saponins complexed to membrane protein antigens (immune stimulating complexes), pluronic polymers with mineral oil, killed mycobacteria in mineral oil, Freund's complete adjuvant, bacterial products, such as muramyl dipeptide (MDP) and lipopolysaccharide (LPS), as well as lipid A, and liposomes. Desirable characteristics of ideal adjuvants may include: (1) lack of toxicity; (2) ability to stimulate a long-lasting immune response; (3) simplicity of manufacture and stability in long-term storage; (4) ability to elicit both CMI and HIR to antigens administered by various routes; (5) synergy with other adjuvants; (6) capability of selectively interacting with populations of antigen presenting cells (APC); (7) ability to specifically elicit appropriate T-cell helper 1 (TH 1) or TH 2 cell-specific immune responses; and (8) ability to selectively increase appropriate antibody isotype levels (for example, IgA) against antigens. An adjuvant used with the present compositions and methods need not possess all these characteristics to be used.

As used herein, “immunogenically effective amount” refers to an amount, which is effective in reducing, eliminating, treating, preventing or controlling the symptoms of the infections, diseases, disorders, or condition.

The quantity to be administered depends on the subject to be treated, including, for example, the capacity of the immune system of the individual to mount a protective response. Suitable regimes for initial administration and booster doses are also variable but may include an initial administration followed by subsequent administrations. For example, it may be desirable to provide for an initial administration of the vaccine followed by additional doses. The need to provide an effective amount of the protective molecule will also need to be balanced with cost of providing higher amounts of the protective molecule. A cost-effective vaccine is one in which the cost of producing it is less than the value one can obtain from using it. Measurement and determination of efficacy of any of the compositions and vaccines of the invention may be accomplished by any of the many methods available to one skilled in the art.

In one embodiment, a straightforward and quick method can be to perform a Western blot analysis of a sample candidate vaccine composition to quantitate the amount of polypeptide or fragment thereof in the sample. In one embodiment, one compares the amount of polypeptide to a standard known to be effective with like polypeptides from other biotypes, and either prepares a vaccine where the level of polypeptide produced is at least at this standard or higher or may test the vaccine with a test animal.

The compounds described herein can be administered to a subject at therapeutically effective doses to prevent SARS-COV-2-associated diseases. The dosage will depend upon the subject receiving the vaccine as well as factors such as the size, weight, and age of the subject.

The precise amount of immunogenic composition of the invention to be employed in a formulation will depend on the route of administration and the nature of the subject (e.g., age, size, stage/level of disease), and should be decided according to the judgment of the practitioner and each subject's circumstances according to standard clinical techniques. An effective immunizing amount is that amount sufficient to treat or prevent a SARS-CoV-2infectious disease in a subject.

Immunogenicity of a composition can be determined by monitoring the immune response of test subjects following immunization with the composition by use of any immunoassay known in the art. Generation of a humoral (antibody) response and/or cell-mediated immunity may be taken as an indication of an immune response.

The immune response of the test subjects can be analyzed by various approaches such as: the reactivity of the resultant immune serum to the immunogenic conjugate, as assayed by known techniques, e.g., enzyme linked immunosorbent assay (ELISA), immunoblots, immunoprecipitations, virus neutralization, etc.; or, by protection of immunized hosts from infection by the pathogen and/or attenuation of symptoms due to infection by the pathogen in immunized hosts as determined by any method known in the art, for assaying the levels of an infectious disease agent, e.g., the viral levels (for example, by culturing of a sample from the subject), or other technique known in the art. The levels of the infectious disease agent may also be determined by measuring the levels of the antigen against which the immunoglobulin was directed. A decrease in the levels of the infectious disease agent or an amelioration of the symptoms of the infectious disease indicates that the composition is effective.

The therapeutics of the invention can be tested in vitro for the desired therapeutic or prophylactic activity, prior to in vivo use. For example, in vitro assays that can be used to determine whether administration of a specific therapeutic is indicated include in vitro cell culture assays in which appropriate cells from a cell line or cells cultured from a subject having a particular disease or disorder are exposed to or otherwise administered a therapeutic, and the effect of the therapeutic on the cells is observed.

Alternatively, the therapeutics may be assayed by contacting the therapeutic to cells (either cultured from a subject or from a cultured cell line) that are susceptible to infection by the infectious disease agent but that are not infected with the infectious disease agent, exposing the cells to the infectious disease agent, and then determining whether the infection rate of cells contacted with the therapeutic was lower than the infection rate of cells not contacted with the therapeutic. Infection of cells with an infectious disease agent may be assayed by any method known in the art.

In addition, the therapeutics can be assessed by measuring the level of the molecule against which the antibody is directed in the animal model and/or human subject at suitable time intervals before, during, or after therapy. Any change or absence of change in the amount of the molecule can be identified and correlated with the effect of the treatment on the subject. The level of the molecule can be determined by any method known in the art.

After vaccination of an animal to SARS-CoV-2 using the methods and compositions of the present invention, any binding assay known in the art can be used to assess the binding between the resulting antibody and the particular molecule. These assays may also be performed to select antibodies that exhibit a higher affinity or specificity for the particular antigen. As one measure of vaccine potency, an ELISA can be performed on a sample collected from an individual vaccinated to determine whether antibodies to a vaccine comprising the sequence, a derivative, a homologue or a variant or fragment thereof generated anti-polypeptide antibodies. The individual's sample is measured against a reference anti-polypeptide antibody. Analysis of symptoms and measurement of animal weight gain also demonstrated lessening of impact of the disease in the presence of a particular dose. Fluorescent focused neutralization assay is still another assay to detect serum neutralizing antibodies and analyze effectiveness of a vaccine and a particular dose.

When testing animals administered the vaccine, for example, measuring antibody response is also effective in determining efficacy of the vaccine. Sera may be collected and titer measured as the reciprocal of the maximal dilution at which hemagglutination is inhibited, as described in an example below. Other measurements post-administration of the vaccine can also be employed to determine effectiveness, whether pathological evaluation, isolation of the pathogen, measurement of symptoms, and overall health and weight gain of the subject.

Thus, the effectiveness of the present vaccine may also be evaluated quantitatively (for example, a decrease in the percentage of diseased tissue as compared to an appropriate control group) or qualitatively (e.g., isolation of virus from blood, detection of virus antigen in a tissue sample by an assay method, etc.). The symptoms of the disease may be evaluated quantitatively (e.g., temperature/fever), semi-quantitatively (e.g., severity of distress, or qualitatively (e.g., the presence or absence of one or more symptoms or a reduction in severity of one or more symptoms,). Clearly one skilled in the art has many different options available for measuring effectiveness of the vaccine. Protection periods of more than seven days after at least one challenge or exposure to the pathogenic microorganism have been achieved, and protection of at least two weeks, at least 20 days, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 days or more, have been achieved using the invention. Such protection periods are also provided when using the invention with other animals. The protective response is also shown here in an embodiment to be specific to the disease as opposed to another disease, and thus demonstrates specific memory.

The vaccine can be administered any convenient method including intranasal, oral and/or parenteral (e.g., intramuscular) administration. For example, the Spike SARS-CoV-2 containing vaccine can be administered intramuscularly one or more times. In another embodiment of the method, for example, the vaccine is administered orally one or more times. In an alternative embodiment oral administration can be followed by and/or precede administration of the vaccine at least once, intramuscularly. The maize grain can be made into a food product and fed to the animal, thereby reducing cost and loss of antigen that can occur through further processing. The following is provided by way of illustration within intending to be limiting of the scope of the invention. All references cited herein are incorporated herein by reference.

### EXAMPLES

### Example 1 (Prophetic)

Create molecular constructs. The S proteins from the coronaviruses of TGEV and PEDV that we have previously expressed in maize grain have a similar structure to the S protein of SARS-CoV-2 with an extensive N-terminal extracellular domain and much smaller C-terminal transmembrane and intracellular domains⁵⁰⁻⁵⁵. In the transmembrane and adjacent regions, an aromatic region and two cysteine boxes important for membrane fusion are conserved. The S1 domain includes the receptor binding domain known to be important for fusion between the viral and host cell membranes. The S1 domain also contains heptad repeat regions (HR1 and HR2) involved in formation of a trimeric fusion core⁸³. The deletion of the transmembrane and intracellular domains helps to stabilize the protein. Thus, previous constructs expressing proteins have been prepared with a truncated version containing the S1 domain with success^{84,85} including a plant-produced S protein⁸⁶ and similar to what we developed for TGEV and PEDV in maize.

A similar approach to express the Si domain for SARS-CoV-2 can be taken. The reference sequence (YP_009724390.1) in GenBank for SARS-CoV-2 S protein is optimized based on the codon bias for maize in a manner similar to our previously published work⁵⁷. Constructs can encode the S1 subdomain (aa 13-685). Additional variants can be made in a similar manner that incorporate the changes. The open reading frames are linked to embryo-preferred promoters in a manner similar to our previously published work^{87,88} that confers high expression with a PIN 2 terminator sequence⁸⁹. The S protein can have a targeting signal directed to the apoplast vacuole or endoplasmic reticulum (ER) as described previously⁹⁰. These have been the most consistent cellular locations for accumulating antigens in maize grain. In some cases, enhancements to the immune response have occurred by fusion of a carrier protein such as the dendritic binding peptide DC3⁹¹ or the LTB peptide⁹². We have shown that the PEDV-S antigen can be fused with these peptides and expresses at levels similar to the S protein alone and maintains immunoreactivity. Therefore, additional expression cassettes are made to include the S antigen fused with these two carrier proteins. These can be targeted to the ER and the apoplast.

These expression cassettes along with an herbicide resistance gene can be used to transform into Agrobacterium and then to transform maize as previously described⁸⁷. In brief, a binary vector system for Agrobacterium will be employed initially and then transferred to an Agrobacterium strain capable of transforming maize. Transformed cell lines will be identified by herbicide resistance and allowed to mature into plants. Expression of individual transformation events in plants has been shown to vary widely among independent events; hence, we target many events per construct and a minimum of 6 plants per event to mature and set seed.

Expression levels of the SARS-CoV-2 S protein in seed can be performed by ELISA in a manner similar to that previously described⁶⁸. In brief, individual seeds from each plant will be extracted separately in PBS and the protein content in the extract measured by the Bradford method. The equivalent of 1 μg of extracted protein can then be tested in an ELISA (S protein and anti-S antibodies are commercially available). Purified S protein can be used as a standard and control seed extracts as a negative control. Six individual seeds per plant can be analyzed and the level of expression in positive seeds used to calculate the mean antigen expression level in each plant that in turn can be used to calculate the mean expression level of each event and finally used to calculate the mean expression of the various constructs. The means can be used to select the constructs and the transformation events with the highest expression. This procedure has been statistically analyzed previously and shown to be sufficient for the selection of the highest expressing lines.

Western blots using anti-S antibodies can be run on selected high expressing lines to confirm the size of the antigen and that it is immunoreactive with the S protein. Next, Western gels can be run using anti-LTB (in-house supply) or anti-DC3 antibody (commercially available) to confirm the fusion protein has not been cleaved by identifying the same size band as the anti-S antibody.

Prepare material for further testing. Selected lines with the highest level of S protein from the constructs can be used for testing the S protein alone as well as the S:LTB and S:DC.

The seed can be propagated to select for high expressing stable lines with no unintended plant alterations. Material from subsequent generations can be used for the studies described below. Grain containing the S antigen can be used to purify the antigen using an antibody affinity column as described previously⁵⁹. In brief, commercially available anti-S IgG will be attached to cyanogen bromide-activated Sepharose beads which can then be used to make a column. The column will be equilibrated with PBS and extracts loaded onto the column, washed with PBS and eluted with high salt at pH3. The eluate run on Western blots to confirm immunoreactivity and on Coomassie-stained gels to obtain an estimate of purity. In addition to Western blots, n-terminal sequence, lectin binding to confirm glycosylation and spectrophotometric analysis can be used to compare the plant-produced protein with that of commercially available S proteins produced from other recombinant hosts. The purified protein may also be used for parenteral administrations.

Grain from lines containing S, S:DC3 and S:LTB can be processed and used to form wafers as described previously⁴⁸. In brief, the grain will be run using our customized processing equipment to enrich for the germ fraction, dried, ground and defatted using SFE. The flour mixed with an equal amount of sucrose and formed into 5 g wafers. Initial target for each wafer is to contain 0.25 mg of the S protein and control flour used for blending if needed.

### Evaluate Different Delivery Methods to Elicit Anti-S Antibodies.

Humanized mice expressing the ACE2 receptor can be presented maize-produced vaccine candidates in wafers (0.25 mg/wafer) for oral delivery or intramuscular injections from the purified S antigen (10 μg plus alum). Antibody titers can be measured from sera and feces sampled over time.

In brief Mice will be divided into 6 groups consisting of: 1) parenterally delivered S, 2) orally delivered wafers containing S, 3) orally delivered wafers containing S fused to LTB, 4) orally delivered wafers containing S fused to DC3, 5) parenterally injected S and boosted with orally delivered S wafers, and 6) parenterally injected S co-administered with orally delivered S wafers. Injections will be given on days 1, 21 and 35. Animals will be fasted overnight then housed individually and provided 2 wafers/mouse (0.5 mg antigen) on the same days as parenteral administration plus the two successive days following the injected dose. The amount consumed will be visually estimated each night and uneaten wafers will be removed. Based on past experience, we anticipate mice will consume 7 g/day (equivalent to 1½ wafers).

Analysis. Sera and fecal samples will be collected as described earlier². In brief, blood will be collected from mice on days 0, 21, 35 and 49. An equal volume of glycerol will be added to all sera samples and will be kept at −20° C. until ready for analysis. Fecal samples will be collected prior to administration of antigens on days 0, 21, 28, 35, 42 and 49. All samples will be kept at −20° C.until ready for analysis. ELISAs will be used to detect anti-S specific antibodies in fecal and sera samples similar to that described previously¹². In brief, purified S will be used to coat the plate followed by the sera or fecal extracts. After washing the plates, an anti-mouse antibody conjugated to alkaline phosphatase will be used for detection. Commercial anti-S antibody will be used as a positive control and titers of individual mice will be compared to their pre-immune sera. Titers showing greater than 4-fold increase over pre-immune levels will be considered to have a positive response. The geometric mean of all mice within a group will be used to determine statistically significant differences between groups by ANOVA and Tukey's HSD procedure, as previously described¹².

Representative samples may be analyzed for neutralizing antibodies (e.g. Creative Diagnostics—see world wide web at creative-diagnostics.com/sars-cov-2-pseudovirus-neturaliztion assay.htm). In brief, the SARS-CoV-2 Spike pseudovirus will be used to infect 293T/ACE2 cells. Viral entry into cells is monitored by the luciferase assay (PVLA). The 293T/ACE2 inhibition of viral entry into cells by neutralizing antibodies is correlated to the decreased levels of luciferase signals in the cells. The percent inhibition at serial dilutions will be evaluated to determine neutralizing activity.

The groups receiving S, S:DC3, and S:LTB will be compared to each other to determine which provides the greatest immune response. If there is no significant difference, S without a carrier protein may be used in subsequent studies. Parenterally delivered S should elicit high titers of sera antibodies but low mucosal antibody titers and the reverse for the orally delivered wafers. We will also determine if 1 or 2 boosts is justified to significantly increase titers. Finally, we will verify that the antibodies have neutralizing activity.

Milestone: Demonstrate that the oral and injected administration regimens can provide an immune response in mice and determine which form of S elicits the strongest immune response.

### Evaluate Protection Conferred by Different Antigen Delivery Methods in a Lethal Murine SARS-CoV-2 Challenge Model.

Humanized mice expressing the ACE2 receptor (hACE2) will be presented the S antigen in wafers for oral delivery and the purified S antigen for parenteral administration. The mice will be challenged intranasally with SARS-CoV-2 and clinical symptoms and infectious viral titers measured similar to procedure outlined previously (Nature Vol 586|22 Oct. 2020 m509.)

The primary measures of efficacy will include: 1) reduced viral shedding in mucosal swabs and BAL, 2) reduced clinical signs and radiographic scores, 3) reduced amounts of virus in tissues collected at necropsy, and 4) reduced histopathology scoring of lungs. Secondary measures of efficacy will include: 1) reduced systemic immune activation/inflammation, and 2) reduced histopathology of other organ systems.

### Conclusions

We have developed a vaccine platform that has the potential to create an orally delivered, low-cost, heat stable, rapidly scalable COVID-19 vaccine. To support this premise, we have demonstrated the following: 1) expression of over 50 different recombinant proteins with some of the highest levels reported including the S protein from SARS-CoV-2, 2) cost and scale-up advantages resulting in commercialization of several purified proteins, 3) oral delivery of several maize-produced vaccine candidates elicited neutralizing antibodies and provided protection for a variety of different pathogens including two different coronaviruses, 4) additive and synergistic effects using a combination of oral and injected delivery methods and 5) heat stability of oral vaccine candidates for at least 1 year at 40° C.

We are now in a position to employ our efforts towards developing a vaccine for COVID-19 that can be orally delivered or used in combination with a parenteral delivered vaccine to provide a highly efficacious vaccine that may be used for groups most vulnerable to COVID-19. Furthermore, production can be scaled to billions of doses in one year starting with only one ear of corn and at a fraction of the cost of traditionally produced vaccines. A heat-stable oral vaccine would also eliminate the need for the cold chain and medical personnel for administration enabling an easier and faster route towards mass immunization of the global population.

### REFERENCES

[1] Szucs, T. (2000) Cost-benefits of vaccination programs, Vaccine 18, S49-S51.
[2] Saplakoglu, Y. (2020) How will the coronavirus outbreak end?, In Live Science.
[3] Ott, J., Stevens, G., Groeger, J., and Wiersma, S. (2012) Global epidemiology of hepatitis B virus infection: new estimates of age-specific HBsAg seroprevalence and endemicity, Vaccine 30, 2212-2219.
[4] Ziesenitz, V. C., Mazer-Amirshahi, M., Zocchi, M. S., Fox, E. R., and May, L. S. (2017) US vaccine and immune globulin product shortages, 2001-15, American Journal of Health-System Pharmacy 74, 1879-1886.
[5] Ecker, D. M., and Ransohoff, T. C. (2013) Mammalian cell culture capacity for biopharmaceutical manufacturing, In Mammalian Cell Cultures for Biologics Manufacturing, pp 185-225, Springer.
[6] Ulmer, J. B., Valley, U., and Rappuoli, R. (2006) Vaccine manufacturing: challenges and solutions, Nature biotechnology 24, 1377-1383.
[7] Keating, G. M., and Noble, S. (2003) Recombinant hepatitis B vaccine (Engerix-B®), Drugs 63, 1021-1051.
[8] Biswas, A., Chakrabarti, A. K., and Dutta, S. (2020) Current challenges: from the path of “original antigenic sin” towards the development of universal flu vaccines: Flu vaccine efficacy encounters significant hurdles from pre-existing immunity of the host suggesting assessment of host immunity before vaccination, International Reviews of Immunology 39, 21-36.
[9] Ahishali, E., Bortas, G., Akyuz, F., Ibrisim, D., Poturoglu, S., Pinarbasi, B., Ozdil, S., and Mungan, Z. (2008) Response to hepatitis B vaccination in patients with celiac disease, Digestive diseases and sciences 53, 2156-2159.
[10] Leonardi, S., Spina, M., Spicuzza, L., Rotolo, N., and La Rosa, M. (2009) Hepatitis B vaccination failure in celiac disease: Is there a need to reassess current immunization strategies?, Vaccine 27, 6030-6033.
[11] Vida Pérez, L., Gómez Camacho, F., Garcia Sánchez, V., Iglesias Flores, E. M., Castillo Molina, L., Cerezo Ruiz, A., Casáis Juanena, L., and De Dios Vega, J. F. (2009) Eficacia de la vacuna contra el virus de la hepatitis B en pacientes con enfermedad inflamatoria intestinal, Medicina clinica 132, 331-335.
[12] Roome, A. J., Walsh, S. J., Cartter, M. L., and Hadler, J. L. (1993) Hepatitis B vaccine responsiveness in Connecticut public safety personnel, JAMA: the journal of the American Medical Association 270, 2931.
[13] Vande Berg, B. J., Hammer, P. E., Chun, B. L., Schouten, L. C., Carr, B., Guo, R., Peters, C., Hinson, T. K., Beilinson, V., Shekita, A., Deter, R., Chen, Z., Samoylov, V., Bryant, C. T., Jr., Stauffer, M. E., Eberle, T., Moellenbeck, D. J., Carozzi, N. B., Koziel, M. G., and Duck, N. B. (2008) Characterization and plant expression of a glyphosate-tolerant enolpyruvylshikimate phosphate synthase, Pest Manag Sci 64, 340-345.
[14] Tohme, R. A., Awosika-Olumo, D., Nielsen, C., Khuwaja, S., Scott, J., Xing, J., Drobeniuc, J., Hu, D. J., Turner, C., and Wafeeg, T. (2011) Evaluation of hepatitis B vaccine immunogenicity among older adults during an outbreak response in assisted living facilities, Vaccine 29, 9316-9320.
[15] Li, L. q., Huang, T., Wang, Y. q., Wang, Z. p., Liang, Y., Huang, T. b., Zhang, H. y., Sun, W., and Wang, Y. (2020) COVID-19 patients' clinical characteristics, discharge rate, and fatality rate of meta-analysis, Journal of medical virology 92, 577-583.
[16] Hamming, I., Timens, W., Bulthuis, M., Lely, A., Navis, G. v., and van Goor, H. (2004) Tissue distribution of ACE2 protein, the functional receptor for SARS coronavirus. A first step in understanding SARS pathogenesis, The Journal of Pathology: A Journal of the Pathological Society of Great Britain and Ireland 203, 631-637.
[17] Neutra, M. R., and Kozlowski, P. A. (2006) Mucosal vaccines: the promise and the challenge, Nature reviews immunology 6, 148-158.
[18] Mahoney, F. J., Stewart, K., Hu, H., Coleman, P., and Alter, M. J. (1997) Progress toward the elimination of hepatitis B virus transmission among health care workers in the United States, Archives of Internal Medicine 157, 2601.
[19] Simard, E. P., Miller, J. T., George, P. A., Wasley, A., Alter, M. J., Bell, B. P., and Finelli, L. (2007) Hepatitis B Vaccination Coverage Levels Among Healthcare Workers in the United States, 2002-2003, Infection control and hospital epidemiology 28, 783-790.
[20] Rogers, B., Dennison, K., Adepoju, N., Dowd, S., and Uedoi, K. (2010) Vaccine cold chain: part 1. proper handling and storage of vaccine, Aaohn Journal 58, 337-346.
[21] Mandolesi, M., Sheward, D. J., Hanke, L., Ma, J., Pushparaj, P., Vidakovics, L. P., Kim, C., Loré, K., Dopico, X. C., and Coquet, J. M. (2020) SARS-CoV-2 protein subunit vaccination elicits potent neutralizing antibody responses, BioRxiv.
[22] Tian, J.-H., Patel, N., Haupt, R., Zhou, H., Weston, S., Hammond, H., Lague, J., Portnoff, A. D., Norton, J., and Guebre-Xabier, M. (2020) SARS-CoV-2 spike glycoprotein vaccine candidate NVX-CoV2373 elicits immunogenicity in baboons and protection in mice, BioRxiv.
[23] Hassan, A. O., Case, J. B., Winkler, E. S., Thackray, L. B., Kafai, N. M., Bailey, A. L., McCune, B. T., Fox, J. M., Chen, R. E., and Alsoussi, W. B. (2020) A SARS-CoV-2 infection model in mice demonstrates protection by neutralizing antibodies, Cell 182, 744-753. e744.
[24] Howard, J. A., and Hood, E. E. (2016) Commercial Plant-produced Recombinant Protein Products, Springer.
[25] Yusibov, V., Streatfield, S. J., and Kushnir, N. (2011) Clinical development of plant-produced recombinant pharmaceuticals: vaccines, antibodies and beyond, Human vaccines 7, 313-321.
[26] Streatfield, S., and Howard, J. (2003) Plant production systems for vaccines, erv 2, 763-775.
[27] Streatfield, S. J., and Howard, J. A. (2003) Plant production systems for vaccines, Expert Rev Vaccines 2, 763-775.
[28] Grisshammer, R. (2006) Understanding recombinant expression of membrane proteins, Current opinion in biotechnology 17, 337-340.
[29] Hood, E. E., Devaiah, S. P., Fake, G., Egelkrout, E., Teoh, K., Requesens, D. V., Hayden, C., Hood, K. R., Pappu, K. M., and Carroll, J. (2012) Manipulating corn germplasm to increase recombinant protein accumulation, Plant biotechnology journal 10, 20-30.
[30] Hayden, C. A., Egelkrout, E. M., Moscoso, A. M., Enrique, C., Keener, T. K., Jimenez-Flores, R., Wong, J. C., and Howard, J. A. (2012) Production of highly concentrated, heat-stable hepatitis B surface antigen in maize, Plant biotechnology journal 10, 979-984.
[31] Streatfield, S., Lane, J., Brooks, C., Barker, D., Poage, M., Mayor, J., Lamphear, B., Drees, C., Jilka, J., and Hood, E. (2003) Corn as a production system for human and animal vaccines, Vaccine 21, 812-815.
[32] Hayden, C. A. (2014) An oral vaccine for hepatitis B: challenges, setbacks, and breakthroughs, In Commercial Plant-Produced Recombinant Protein Products, pp 197-228, Springer.
[33] Egelkrout, E., Hayden, C., Fake, G., Keener, T., Arruda, P., Saltzman, R., Walker, J., and Howard, J. (2020) Oral delivery of maize-produced porcine epidemic diarrhea virus spike protein elicits neutralizing antibodies in pigs, Plant Cell, Tissue and Organ Culture, 1.
[34] Streatfield, S., and Howard, J. (2003) Plant-based vaccines, International journal for parasitology 33, 479-493.
[35] Fischer, R., Stoger, E., Schillberg, S., Christou, P., and Twyman, R. M. (2004) Plant-based production of biopharmaceuticals, Curr Opin Plant Biol 7, 152-158.
[36] Yuki, Y., and Kiyono, H. (2009) Mucosal vaccines: novel advances in technology and delivery, Expert review of vaccines 8, 1083-1097.
[37] Agnihotri, N., Mishra, R., Goda, C., and Arora, M. (2012) Microencapsulation—a novel approach in drug delivery: a review, Indo Global Journal of Pharmaceutical Sciences 2, 1-20.
[38] Dai, C., Wang, B., and Zhao, H. (2005) Microencapsulation peptide and protein drugs delivery system, Colloids and Surfaces B: Biointerfaces 41, 117-120.
[39] Bailey, M. R. (2000), Texas A&M University.
[40] Arango, I. P., Rubio, E. L., Anaya, E. R., Flores, T. O., de la Vara, L. G., and Lim, M. A. G. (2008) Expression of the rabies virus nucleoprotein in plants at high-levels and evaluation of immune responses in mice, Plant cell reports 27, 677-685.
[41] Loza-Rubio, E., Rojas, E., Gomez, L., Olivera, M., and Gomez-Lim, M. (2007) Development of an edible rabies vaccine in maize using the Vnukovo strain, Developments in biologicals 131, 477-482.
[42] Loza-Rubio, E., Rojas-Anaya, E., López, J., Olivera-Flores, M. T., Gómez-Lim, M., and Tapia-Pérez, G. (2012) Induction of a protective immune response to rabies virus in sheep after oral immunization with transgenic maize, expressing the rabies virus glycoprotein, Vaccine 30, 5551-5556.
[43] Modelska, A., Dietzschold, B., Sleysh, N., Fu, Z. F., Steplewski, K., Hooper, D. C., Koprowski, H., and Yusibov, V. (1998) Immunization against rabies with plant-derived antigen, Proceedings of the National Academy of Sciences 95, 2481-2485.
[44] Yusibov, V., Hooper, D., Spitsin, S., Fleysh, N., Kean, R., Mikheeva, T., Deka, D., Karasev, A., Cox, S., and Randall, J. (2002) Expression in plants and immunogenicity of plant virus-based experimental rabies vaccine, Vaccine 20, 3155-3164.
[45] Guerrero-Andrade, O., Loza-Rubio, E., Olivera-Flores, T., Fehervari-Bone, T., and Gomez-Lim, M. A. (2006) Expression of the Newcastle disease virus fusion protein in transgenic maize and immunological studies, Transgenic Res 15, 455-463.
[46] Guerrero-Andrade, O., Loza-Rubio, E., Olivera-Flores, T., Fehérvári-Bone, T., and Gómez-Lim, M. A. (2006) Expression of the Newcastle disease virus fusion protein in transgenic maize and immunological studies, Transgenic Research 15, 455-463.
[47] Yang, Z. Q., Liu, Q. Q., Pan, Z. M., Yu, H. X., and Jiao, X. A. (2007) Expression of the fusion glycoprotein of Newcastle disease virus in transgenic rice and its immunogenicity in mice, Vaccine 25, 591-598.
[48] Hayden, C. A., Smith, E. M., Turner, D. D., Keener, T. K., Wong, J. C., Walker, J. H., Tizard, I. R., Jimenez-Flores, R., and Howard, J. A. (2014) Supercritical fluid extraction provides an enhancement to the immune response for orally-delivered hepatitis B surface antigen, Vaccine 32, 1240-1246.
[49] Lamphear, B. J., Jilka, J. M., Kesl, L., Welter, M., Howard, J. A., and Streatfield, S. J. (2004) A corn-based delivery system for animal vaccines: an oral transmissible gastroenteritis virus vaccine boosts lactogenic immunity in swine, Vaccine 22, 2420-2424.
[50] Lamphear, B. J., Streatfield, S. J., Jilka, J. M., Brooks, C. A., Barker, D. K., Turner, D. D., Delaney, D. E., Garcia, M., Wiggins, B., and Woodard, S. L. (2002) Delivery of subunit vaccines in maize seed, Journal of Controlled Release 85, 169-180.
[51] Bootland, L., and Beifuss, K. (2011) Immunization of fish with plant-expressed recombinant proteins, Google Patents.
[52] Bootland, L., and Beifuss, K. (2008) Immunization of fish with plant-expressed recombinant proteins, Google Patents.
[53] Streatfield, S., Jilka, J., Hood, E., Turner, D., Bailey, M., Mayor, J., Woodard, S., Beifuss, K., Horn, M., and Delaney, D. (2001) Plant-based vaccines: unique advantages, Vaccine 19, 2742-2748.
[54] Aldwell, F., Cross, M., Fitzpatrick, C., Lambeth, M., De Lisle, G., and Buddle, B. (2006) Oral delivery of lipid-encapsulated Mycobacterium bovis BCG extends survival of the bacillus in vivo and induces a long-term protective immune response against tuberculosis, Vaccine 24, 2071-2078.
[55] Aldwell, F. E., Keen, D. L., Parlane, N. A., Skinner, M. A., de Lisle, G. W., and Buddle, B. M. (2003) Oral vaccination with Mycobacterium bovis BCG in a lipid formulation induces resistance to pulmonary tuberculosis in brushtail possums, Vaccine 22, 70-76.
[56] Arlen, P. A., Singleton, M., Adamovicz, J. J., Ding, Y., Davoodi-Semiromi, A., and Daniell, H. (2008) Effective plague vaccination via oral delivery of plant cells expressing F1-V antigens in chloroplasts, Infect Immun 76, 3640-3650.
[57] Tacket, C., Pasetti, M., Edelman, R., Howard, J., and Streatfield, S. (2004) Immunogenicity of recombinant LT-B delivered orally to humans in transgenic corn, Vaccine 22, 4385-4389.
[58] Hayden, C. A., Landrock, D., Hung, C. Y., Ostroff, G., Fake, G. M., Walker, J. H., Kier, A., and Howard, J. A. (2020) Co-Administration of Injected and Oral Vaccine Candidates Elicits Improved Immune Responses over Either Route Alone, Vaccines 8, 37.
[59] Shah, S., Hayden, C. A., Fischer, M. E., Rao, A. G., and Howard, J. A. (2015) Biochemical and biophysical characterization of maize-derived HBsAg for the development of an oral vaccine, Archives of biochemistry and biophysics 588, 41-49.
[60] Fischer, R., Stoger, E., Schillberg, S., Christou, P., and Twyman, R. M. (2004) Plant-based production of biopharmaceuticals, Current opinion in plant biology 7, 152-158.
[61] Lamphear, B. J., Streatfield, S. J., Jilka, J. M., Brooks, C. A., Barker, D. K., Turner, D. D., Delaney, D. E., Garcia, M., Wiggins, B., Woodard, S. L., Hood, E. E., Tizard, I. R., Lawhorn, B., and Howard, J. A. (2002) Delivery of subunit vaccines in maize seed, J Control Release 85, 169-180.
[62] Streatfield, S. J., Lane, J. R., Brooks, C. A., Barker, D. K., Poage, M. L., Mayor, J. M., Lamphear, B. J., Drees, C. F., Jilka, J. M., Hood, E. E., and Howard, J. A. (2003) Corn as a production system for human and animal vaccines, Vaccine 21, 812-815.
[63] Hayden, C. A., Fischer, M. E., Andrews, B. L., Chilton, H. C., Turner, D. D., Walker, J. H., Tizard, I. R., and Howard, J. A. (2015) Oral delivery of wafers made from HBsAg-expressing maize germ induces long-term immunological systemic and mucosal responses, Vaccine 33, 2881-2886.
[64] Horn, M., Pappu, K., Bailey, M., Clough, R., Barker, M., Jilka, J., Howard, J., and Streatfield, S. (2003) Advantageous features of plant-based systems for the development of HIV vaccines, Journal of drug targeting 11, 539-545.
[65] Horn, M., Woodard, S., and Howard, J. (2004) Plant molecular farming: systems and products, Plant cell reports 22, 711-720.
[66] Howard, J. A., and Hood, E. (2005) Bioindustrial and biopharmaceutical products produced in plants, Advances in agronomy 85, 91-124.
[67] Luna V., S., Figueroa M., J., Baltazar M., B., Gomez L., R., Townsend, R., and Schoper, J. B. (2001) Maize Pollen Longevity and Distance Isolation Requirements for Effective Pollen Control, Crop Sci 41, 1551-1557.
[68] Erlewyn-Lajeunesse, M., Brathwaite, N., Lucas, J., and Warner, J. (2009) Recommendations for the administration of influenza vaccine in children allergic to egg, Bmj 339, b3680.
[69] Loveless, M. H. (1950) Allergy for corn and its derivatives: experiments with a masked ingestion test for its diagnosis, Journal of Allergy 21, 500-509.
[70] Dubois, B., Goubier, A., Joubert, G., and Kaiserlian, D. (2005) Oral tolerance and regulation of mucosal immunity, Cellular and molecular life sciences: CMLS 62, 1322.
[71] Garside, P., and Mowat, A. M. (2001) Oral tolerance, In Seminars in immunology, pp 177-185, Elsevier.
[72] Strobel, S. (1992) Dietary manipulation and induction of tolerance, The Journal of pediatrics 121, S74-S79.
[73] Hayden, C. A., Streatfield, S. J., Lamphear, B. J., Fake, G. M., Keener, T. K., Walker, J. H., Clements, J. D., Turner, D. D., Tizard, I. R., and Howard, J. A. (2012) Bioencapsulation of the hepatitis B surface antigen and its use as an effective oral immunogen, Vaccine 30, 2937-2942.
[74] Landry, N., Ward, B. J., Trépanier, S., Montomoli, E., Dargis, M., Lapini, G., and Vézina, L.-P. (2010) Preclinical and clinical development of plant-made virus-like particle vaccine against avian H5N1 influenza, PLoS One 5, e15559.
[75] Mett, V., Musiychuk, K., Bi, H., Farrance, C. E., Horsey, A., Ugulava, N., Shoji, Y., De La Rosa, P., Palmer, G. A., and Rabindran, S. (2008) A plant-produced influenza subunit vaccine protects ferrets against virus challenge, Influenza and other respiratory viruses 2, 33-40.
[76] Shoji, Y., Bi, H., Musiychuk, K., Rhee, A., Horsey, A., Roy, G., Green, B., Shamloul, M., Farrance, C. E., and Taggart, B. (2009) Plant-derived hemagglutinin protects ferrets against challenge infection with the A/Indonesia/05/05 strain of avian influenza, Vaccine 27, 1087-1092.
[77] Shoji, Y., Chichester, J. A., Bi, H., Musiychuk, K., de la Rosa, P., Goldschmidt, L., Horsey, A., Ugulava, N., Palmer, G. A., and Mett, V. (2008) Plant-expressed HA as a seasonal influenza vaccine candidate, Vaccine 26, 2930-2934.
[78] Shoji, Y., Chichester, J. A., Jones, M., Manceva, S. D., Damon, E., Mett, V., Musiychuk, K., Bi, H., Farrance, C., and Shamloul, M. (2011) Plant-based rapid production of recombinant subunit hemagglutinin vaccines targeting H1N1 and H5N1 influenza, Human vaccines 7, 41-50.
[79] Wigdorovitz, A., Mozgovoj, M., Santos, M. J. D., Parreño, V., Gómez, C., Pérez-Filgueira, D. M., Trono, K. G., Ríos, R. D., Franzone, P. M., and Fernández, F. (2004) Protective lactogenic immunity conferred by an edible peptide vaccine to bovine rotavirus produced in transgenic plants, Journal of general virology 85, 1825-1832.
[80] Wu, Y.-Z., Li, J.-T., Mou, Z.-R., Fei, L., Ni, B., Geng, M., Jia, Z.-C., Zhou, W., Zou, L.-Y., and Tang, Y. (2003) Oral immunization with rotavirus VP7 expressed in transgenic potatoes induced high titers of mucosal neutralizing IgA, Virology 313, 337-342.
[81] Yu, J., and Langridge, W. (2003) Expression of rotavirus capsid protein VP6 in transgenic potato and its oral immunogenicity in mice, Transgenic research 12, 163-169.
[82] Day, C. W., Baric, R., Cai, S. X., Frieman, M., Kumaki, Y., Money, J. D., Smee, D. F., and Barnard, D. L. (2009) A new mouse-adapted strain of SARS-CoV as a lethal model for evaluating antiviral agents in vitro and in vivo, Virology 395, 210-222.
[83] Ma, G., Feng, Y., Gao, F., Wang, J., Liu, C., and Li, Y. (2005) Biochemical and biophysical characterization of the transmissible gastroenteritis coronavirus fusion core, Biochemical and biophysical research communications 337, 1301-1307.
[84] Oh, J., Lee, K.-W., Choi, H.-W., and Lee, C. (2014) Immunogenicity and protective efficacy of recombinant S1 domain of the porcine epidemic diarrhea virus spike protein, Archives of virology 159, 2977-2987.
[85] Li, J., Ulitzky, L., Silberstein, E., Taylor, D. R., and Viscidi, R. (2013) Immunogenicity and protection efficacy of monomeric and trimeric recombinant SARS coronavirus spike protein subunit vaccine candidates, Viral immunology 26, 126-132.
[86] Pogrebnyak, N., Golovkin, M., Andrianov, V., Spitsin, S., Smirnov, Y., Egolf, R., and Koprowski, H. (2005) Severe acute respiratory syndrome (SARS) S protein production in plants: development of recombinant vaccine, Proceedings of the National Academy of Sciences 102, 9062-9067.
[87] Lamphear, B., Barker, D., Brooks, C., Delaney, D., Lane, J., Beifuss, K., Love, R., Thompson, K., Mayor, J., and Clough, R. (2005) Expression of the sweet protein brazzein in maize for production of a new commercial sweetener, Plant biotechnology journal 3, 103-114.
[88] Streatfield, S. J., Bray, J., Love, R. T., Horn, M. E., Lane, J. R., Drees, C. F., Egelkrout, E. M., and Howard, J. A. (2010) Identification of maize embryo-preferred promoters suitable for high-level heterologous protein production, GM crops 1, 162-172.
[89] Xing, A., Moon, B. P., Mills, K. M., Falco, S. C., and Li, Z. (2010) Revealing frequent alternative polyadenylation and widespread low-level transcription read-through of novel plant transcription terminators, Plant biotechnology journal 8, 772-782.
[90] Hood, E., Bailey, M., Beifuss, K., Magallanes-Lundback, M., Horn, M., Callaway, E., Drees, C., Delaney, D., Clough, R., and Howard, J. (2003) Criteria for high-level expression of a fungal laccase gene in transgenic maize, Plant biotechnology journal 1, 129-140.
[91] Mohamadzadeh, M., Duong, T., Sandwick, S. J., Hoover, T., and Klaenhammer, T. R. (2009) Dendritic cell targeting of Bacillus anthracis protective antigen expressed by Lactobacillus acidophilus protects mice from lethal challenge, Proc Natl Acad Sci USA 106, 4331-4336.
[92] O'Dowd, A., Botting, C., Precious, B., Shawcross, R., and Randall, R. (1999) Novel modifications to the C-terminus of LTB that facilitate site-directed chemical coupling of antigens and the development of LTB as a carrier for mucosal vaccines, Vaccine 17, 1442-1453.
[93] Dinnon, K. H., Leist, S. R., Schäfer, A., Edwards, C. E., Martinez, D. R., Montgomery, S. A., West, A., Yount, B. L., Hou, Y. J., and Adams, L. E. (2020) A mouse-adapted model of SARS-CoV-2 to test COVID-19 countermeasures, Nature, 1-9.
[94] Moreau, G. B., Burgess, S. L., Sturek, J. M., Donlan, A. N., Petri Jr, W. A., and Mann, B. J. (2020) Evaluation of K18-hACE2 mice as a model of SARS-CoV-2 infection, The American Journal of Tropical Medicine and Hygiene, tpmd200762.

### Example 2

### Construct Analysis and Details

Four plasmid constructs were prepared as described in Example 1.
Plasmid 9313/construct COA: pr44-BAASS:Cov-S1
Plasmid 9314/construct COB: pr44-BAASS:Cov-S1 KDEL
Plasmid 9315/construct COC: pr44-BAASS:Cov-S1-DC peptide
Plasmid 9316/construct COD: pr44-BAASS:Cov-S1 LTB

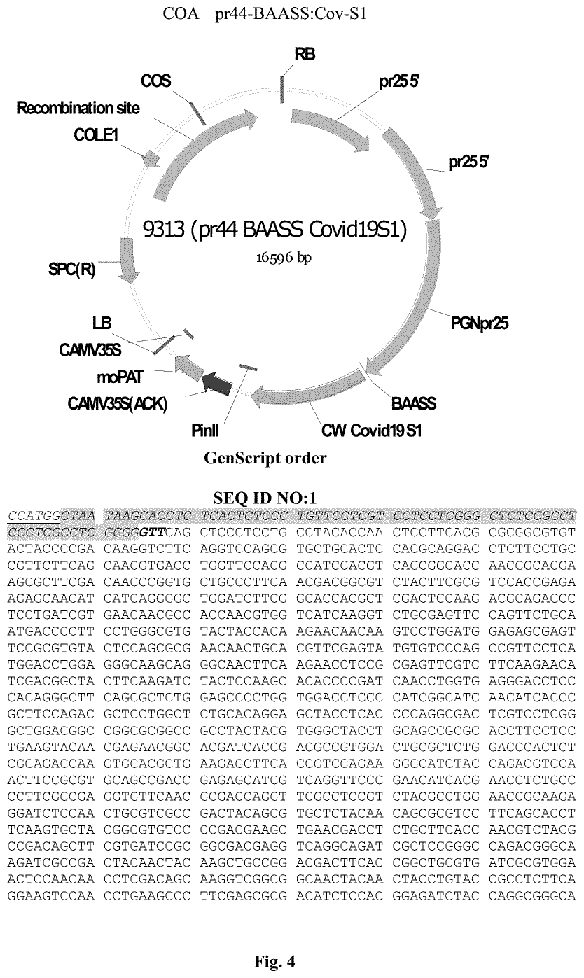

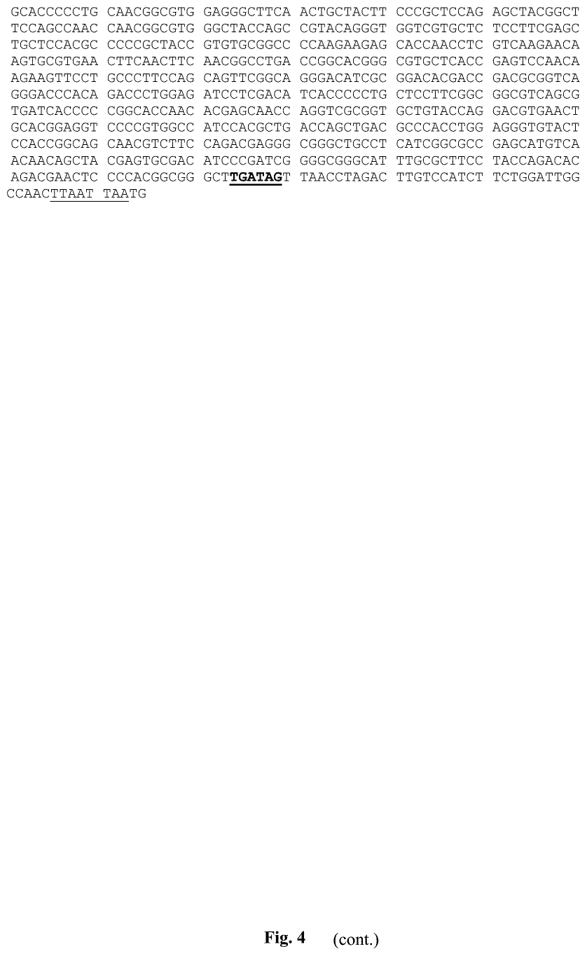

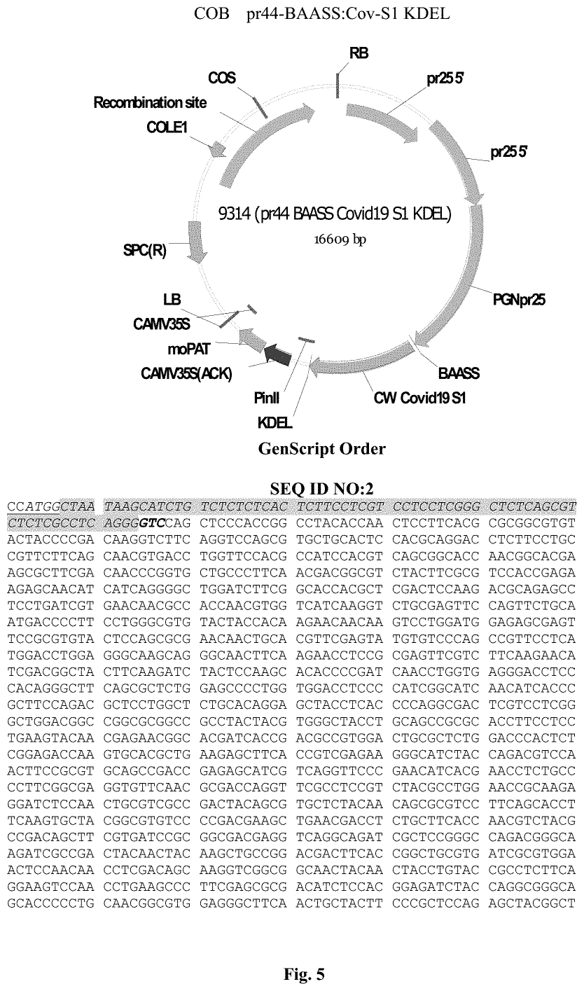

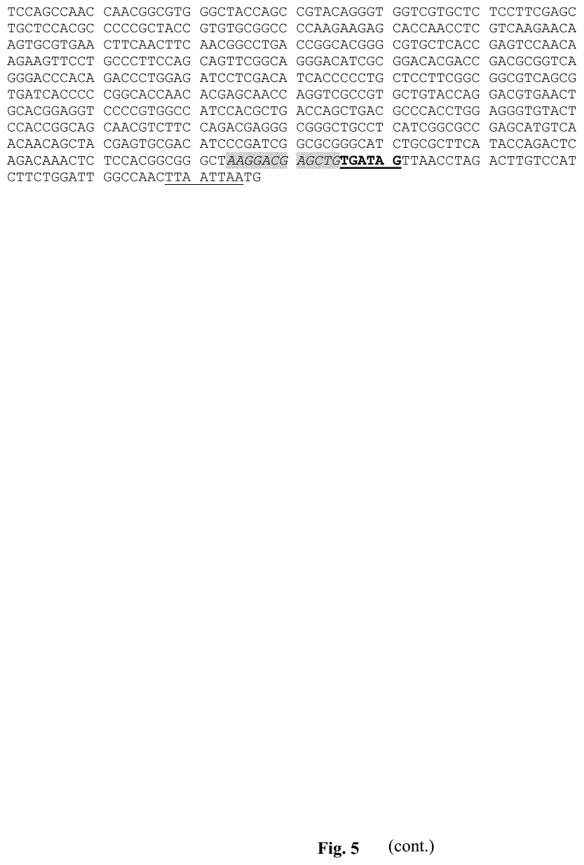

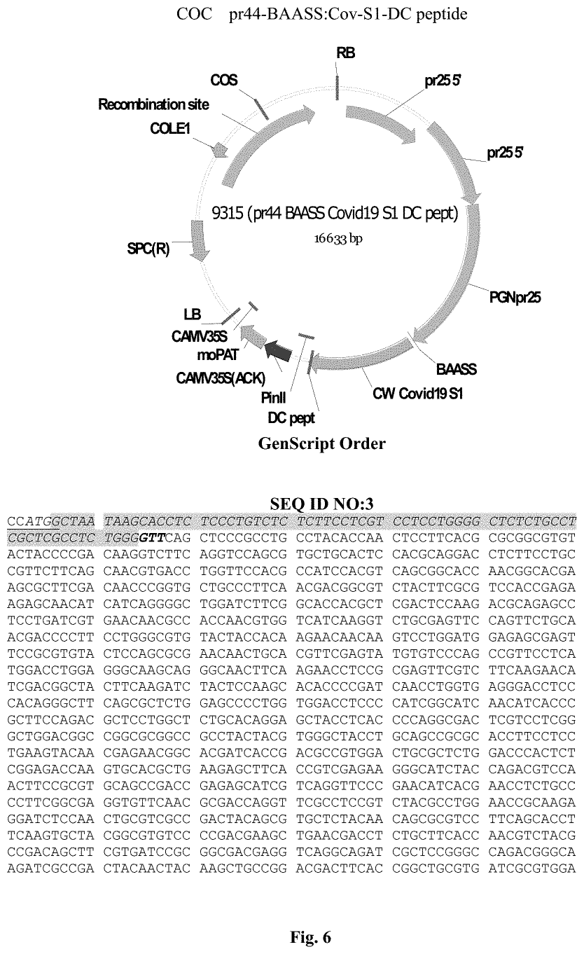

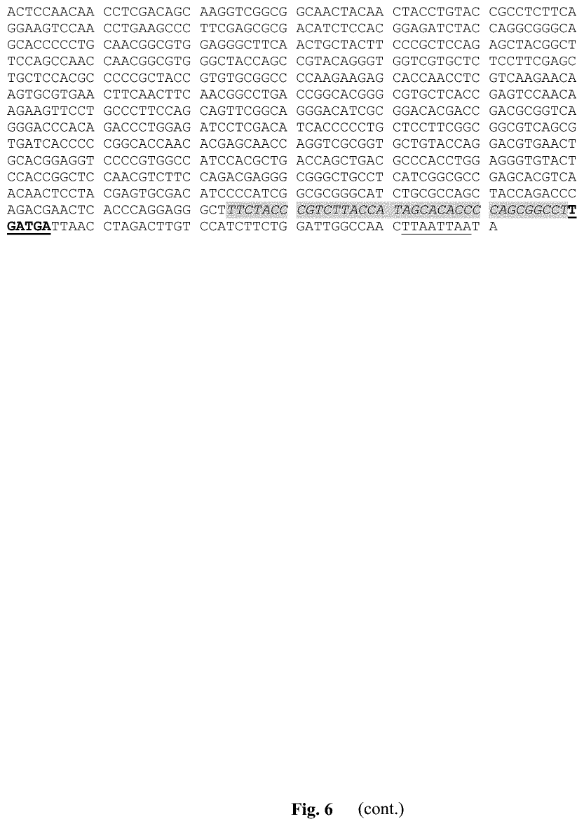

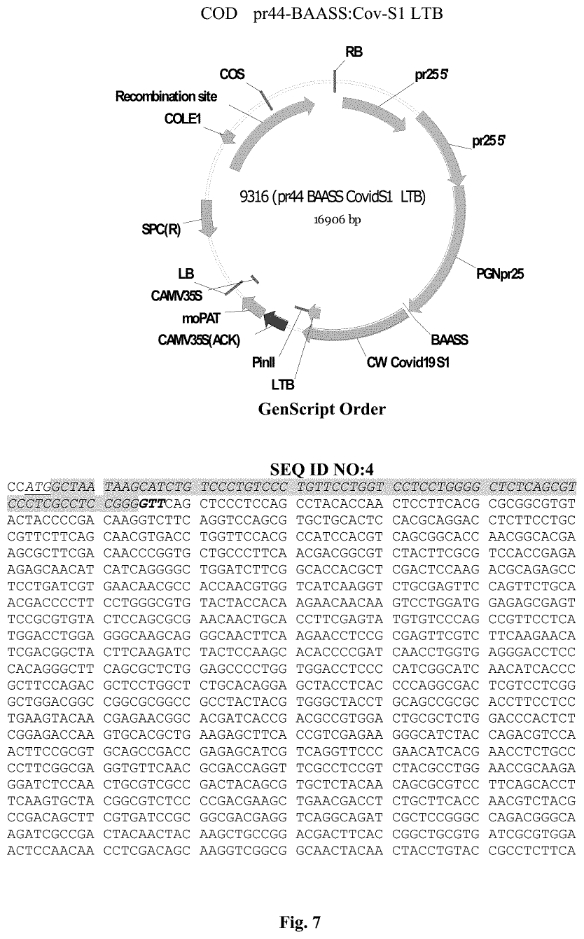

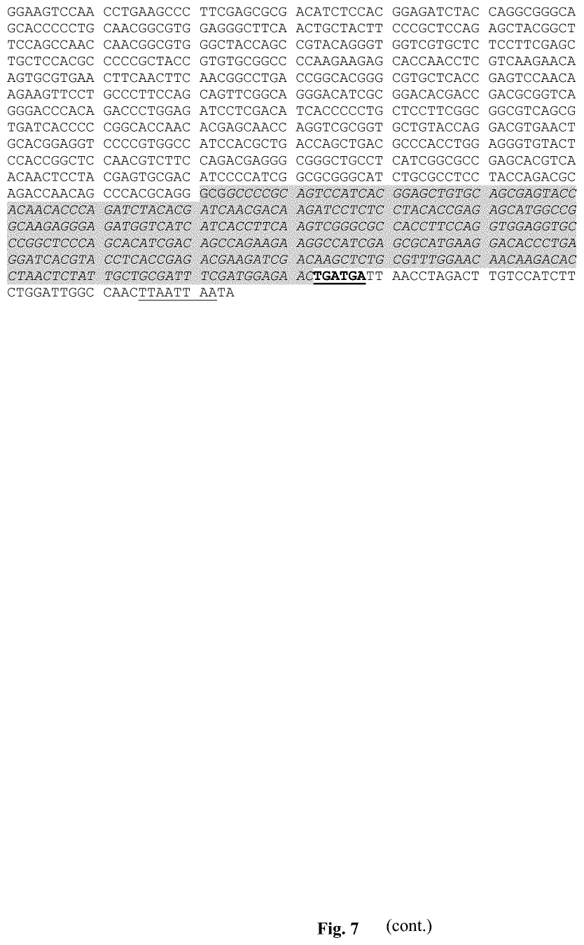

The S1 subunit of the SARS-CoV-2 Spike protein was used as per the FIGS. 4-7 with the native signal sequence replaced by the barley alpha amylase (BAASS) signal sequence. The Covid-19 S1 sequence is from Genbank GCF_009858895.2, S sequence from 23-684 a.a.-5′UTR start in pr25/pr44 defined in Genbank accession L22344.1 (targets the protein to the embryo). A valine was added to predicted N-terminus of Covid-19 S1 to increase N-end stability once signal sequence is cleaved. See FIG. 3. See FIGS. 4-8 for Construct diagrams and sequences of maize-optimized SARS-CoV-2 Spike protein S1 subunit coding region gene fragment synthesized by GenScript.

### Example 3

### Seed S-Protein Analysis

Accumulation of antigen in host. Low levels of antigen accumulation in the host is one of the main factors that have limited commercialization of plant-based vaccines. This not only adds to cost but also eliminates the preferred option of oral delivery that requires much higher doses and restricts the amount that can be administered in a single dose. Furthermore, many subunit vaccine candidates, including the S protein, are membrane-associated, which are notoriously difficult to express in recombinant systems²⁸, making protein accumulation even a greater challenge.

To overcome this limitation, we developed promoters that confer very high levels of recombinant proteins in maize grain (U.S. Pat. Nos. 7,183,109; 7,112,723; 8,642,749). Next, introgression into elite germplasm and selection for high expression across generations was performed. This step has consistently improved the levels of recombinant protein by a minimum of 10-fold from the levels in the first generation. The combination of these two approaches led to some of the highest levels of accumulation of recombinant proteins in plants²⁹.

With constructs that confer expression preferentially targeted to the embryo (germ), enrichment of the germ fraction can be implemented to concentrate the antigen (U.S. Pat. No. 6,504,085) even more. This involves mechanically separating the endosperm (starch) and germ fractions of the seed. Fractionation can concentrate the antigen an additional 7-fold without extracting the protein. We have used this method to successfully concentrate several recombinant proteins in maize grain that led to orders of magnitude higher levels of accumulation for vaccine candidates compared to other plant hosts^{37,38}.

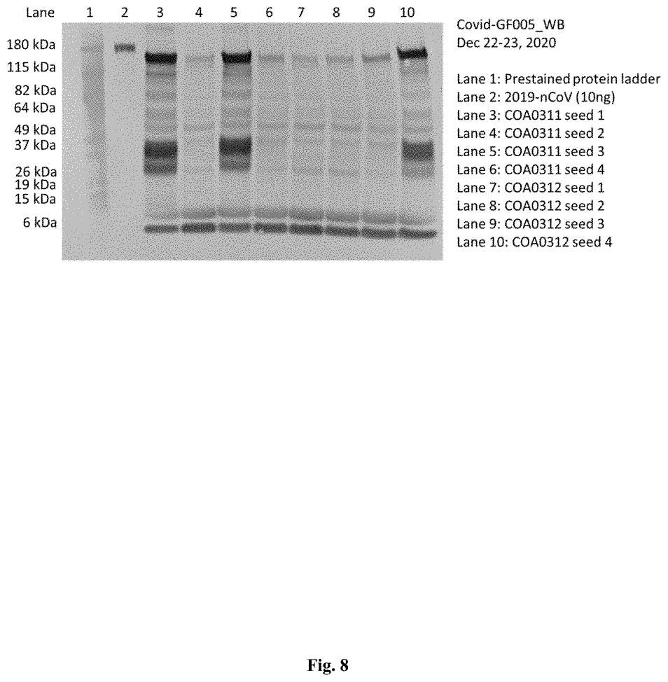

Coronavirus vaccines require the expression of the S protein that accumulates poorly in recombinant hosts. When using the maize platform however, expression of the S protein of the coronavirus PEDV, levels of >20 μg/g were obtained in the initial transformed plants³⁹. We took a similar approach with SARS-CoV-2 and preliminary results indicate that the SARS-CoV-2 S protein can accumulate to >50 μg/g without optimization (FIG. 8). After optimization, we anticipate levels of >1 mg S protein/g enabling a >50 mg dose in one ounce of biomass. This is encouraging as suggests this will not be a rate limiting factor to achieve high levels of VLPs.

### Reagents for Western Blots:

Standard: SARS-CoV-2 (2019-nCoV) Spike Protein (S1 Subunit, His Tag) from Sino Biological, cat #40591-VO8H, reconstituted at 0.25 mg/ml, frozen in 20 ul aliquots. Diluted 1:250 to 0.001 ug/ul.

Ladder: Benchmark pre-stained protein ladder (Invitrogen cat #10748-010)

Samples: 4 seeds of COA0311 (seed wt=76, 105, 61, 93 mg) and 4 seeds of COA0312 (seed wt=92, 97, 75, 98 mg), extracted in 1 ml 1× PBS.

### Primary Ab:

SARS-CoV-2/2019-nCoV Spike/RBD (receptor binding domain) Antibody

Diluted 1:500 (40 ul Ab+20 ml blocking solution)

Secondary Ab: AP-conjugated goat anti-rabbit IgG (Jackson #111-055-003), diluted 1:2,000 (20 ul Ab <1:2 in glycerol>+20 mL block)

Substrate: BCIP/NBT substrate (Sigma #B5655)

Gel type: NuPAGE 4-12% Bis-Tris gel, 150V, 45 min

The sequence for the S protein including the S1 domain was used to transform maize.

Seeds were harvested and analyzed by Western blots to detect for S protein. Lane 2 is the S protein obtained from a commercial supplier. Lanes 3-10 are individual seeds from first generation plants expected to segregate 50% for the gene. Lanes 3, 5 and 10 show specific bands at the expected MW of ˜120 kD. See FIG. 8.

Gel

| | | Amt | Sam- | LDS | Re- | | |
|---|---|---|---|---|---|---|---|
| | [Total | protein | ple | sample | ducing | | |
| | protein] | loaded | vol | buffer | Agent | ddH2O | |
| Sample | (mg/ml) | (ug) | (uL) | (4X) | (ul) | (uL) | Lane |
| Ladder | | | 15 | | | | 1 |
| 2019-nCoV | 0.001 | 0.01 | 10 | 6.3 | 2.5 | 6.2 | 2 |
| COA0311-01 | | | 16.2 | 6.3 | 2.5 | 0 | 3 |
| COA0311-02 | | | 16.2 | 6.3 | 2.5 | 0 | 4 |
| COA0311-03 | | | 16.2 | 6.3 | 2.5 | 0 | 5 |
| COA0311-04 | | | 16.2 | 6.3 | 2.5 | 0 | 6 |
| COA0312-01 | | | 16.2 | 6.3 | 2.5 | 0 | 7 |
| COA0312-01 | | | 16.2 | 6.3 | 2.5 | 0 | 8 |
| COA0312-01 | | | 16.2 | 6.3 | 2.5 | 0 | 9 |
| COA0312-01 | | | 16.2 | 6.3 | 2.5 | 0 | 10 |

Samples heated at 99 C. for 10 min
Loaded ~25 ul per lane

### Example 4

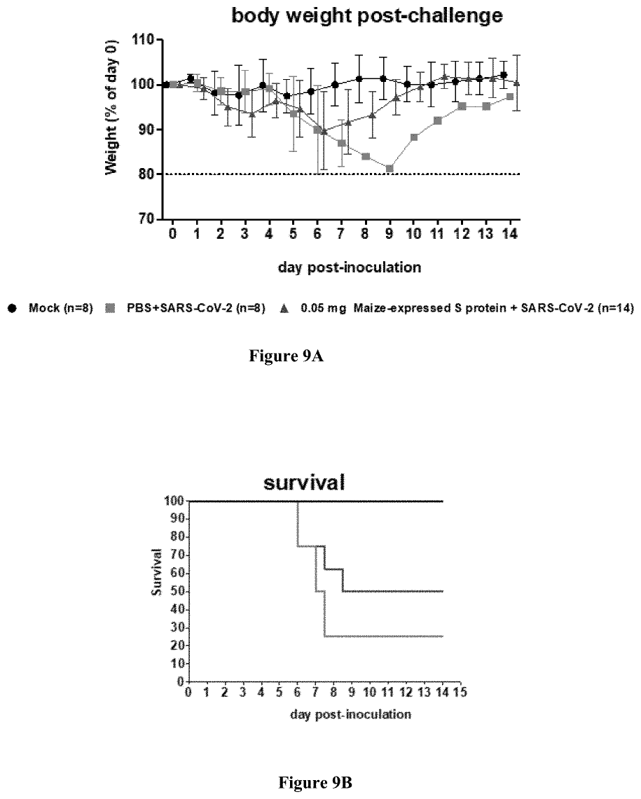

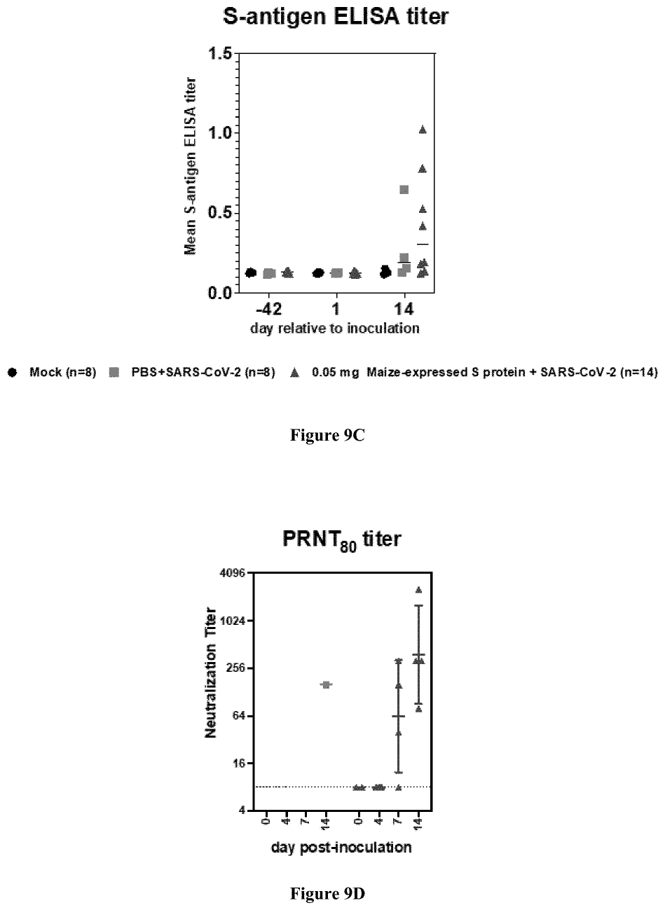

Seed from this material was grown to produce grain that in turn used to provide 2 doses of 150 μg each which was administered to mice orally over 2, 3-day windows at days −42 to −40 and −14 to −12 to young transgenic humanized ACE2 receptor (hACE2) expressing mice⁵⁷. The mice were challenged with a lethal dose of a Delta variant of SARS-CoV-2 12 days after they were last presented maize grain. The results are shown in FIG. 9.

Mice orally administered S protein (blue data points) exhibited less weight loss (FIG. 9A), reduced mortality (FIG. 9B), generated anti-S antibodies (FIG. 9C) and had neutralizing activity in sera (FIG. 9D). One of the non-vaccinated mice (red data points) survived the challenge and had a greater weight loss than the mean of the vaccinated mice for unknown reasons. This one mouse was also the only one that showed an increase in antibody titer and neutralization activity. This demonstrated not only the potential for oral delivery to provide protection from lethal SARS-CoV-2, but also that the S from a WA1-like strain cross protected against the Delta variant SARS-CoV-2.

TABLE OF SEQUENCES

| | |
|---|---|
| SEQ ID NO: 1 | insert in pr44-BAASS:Cov-S1 (COA) |
| SEQ ID NO: 2 | insert in pr44-BAASS:Cov-S1 KDEL (COB) |
| SEQ ID NO: 3 | insert in pr44-BAASS:Cov-S1-DC peptide (COC) |
| SEQ ID NO: 4 | insert in pr44-BAASS:Cov-S1 LTB (COD) |
| SEQ ID NO: 5 | BAASS sequence |
| SEQ ID NO: 6 | BAASS encoded peptide |
| SEQ ID NO: 7 | DC 3 |
| SEQ ID NO: 8 | LTB heat labile peptide |
| SEQ ID NO: 9 | LTB heat labile encoded peptide |
| SEQ ID NO: 10 | Promoter pr25—globulin l |
| SEQ ID NO: 11 | Promoter pr39—27 kDa gamma zein |
| SEQ ID NO: 12 | Promoter P44 sequence |
| SEQ ID NO: 13 | KDEL ER targeting sequence |
| SEQ ID NO: 14 | HDEL sequence |
| SEQ ID NO: 15 | DR 13 |
| SEQ ID NO: 16 | Genbank YP_009724390.1 |
| SEQ ID NO: 17 | COA protein |
| SEQ ID NO: 18 | COB protein |
| SEQ ID NO: 19 | COC protein |
| SEQ ID NO: 20 | COD protein |
| SEQ ID NO: 21 | COA protein without signal sequence |
| SEQ ID NO: 22 | COB protein without signal sequence |
| SEQ ID NO: 23 | COC protein without signal sequence |
| SEQ ID NO: 24 | COD protein without signal sequence |

What is claimed is:

1. A method of producing a protective response to Severe Acute Respiratory Syndrome Coronavirus 2 (SARS-CoV-2) in an animal, the method comprising,
a) orally administering to said animal a composition comprising plant or plant product comprising the Spike (51) protein of SARS-CoV-2, or a sequence having at least 90% identity thereto or a functional fragment thereof
b) producing a protective response to said SARS-CoV-2 in said animal.

2. The method of claim 1 wherein said plant part is seed or embryo of seed.

3. The method of claim 1 wherein said S1 protein expressed at levels of at least 10 mg/kb in seed of said plant.

4. The method of claim 1, wherein said protective response comprises an antibody response in said animal.

5. The method of claim 1, wherein said antibody response is at least 4 times greater than antibody response in an animal not administered said vaccine.

6. The method of claim 1, wherein said composition comprises plant material or plant tissue.

7. The method of claim 1 wherein said 51 protein encoding sequence is optimized for maize expression.

8. A vaccine comprising a plant-produced polypeptide of the Spike (S1) protein, the vaccine comprising a plant or plant product comprising a construct comprising,
(a) a promoter directing expression in a plant cell;
(b) a nucleic acid molecule encoding a S1 polypeptide of said SARS-CoV-2 as disclosed herein, or a sequence having at least 90% identity thereto or a functional fragment operably linked to said promoter; and
(c) a nucleic acid molecule targeting expression of said polypeptide in the endoplasmic reticulum of said plant.

9. The vaccine of claim 8 wherein said promoter preferentially directs expression to the seed of said plant.

10. The vaccine of claim 8 wherein said plant product is seed.

11. The vaccine of claim 9 wherein said Si polypeptide in said plant is expressed at levels of at least 1 mg/kg of seed of said plant.

12. The vaccine of claim 8, wherein said construct further is SEQ ID NO: 1, 2, 3, or 4 or a sequence with 90% homology thereto.

13. The vaccine of claim 8, wherein said construct comprises two copies of said nucleic acid molecule encoding a Si polypeptide.

14. The vaccine of claim 9, wherein said construct comprises pr44-BAASS:Cov-S1, pr44-BAASS:Cov-S1 KDEL, pr44-BAASS:Cov-S1-DC peptide, or pr44-BAASS:Cov-S1 LTB.

15. The vaccine of claim 8, wherein said S1 protein or fragment thereof is expressed in said plant or plant product at levels of 10 mg/kg of whole seed.

16. The vaccine of claim 8, wherein said construct encodes a protein of SEQ ID NO: 17, 18, 19, 20, 21, 22, 23, or 24 or a sequence with 90% homology thereto.

17. The vaccine of claim 8, wherein said Si protein is an amino acid sequence of 21, 22, 23, or 24 or a protein with 90% homology thereto.

18. The vaccine of claim 8 wherein said S1 protein is purified from said plant or plant part to form a vaccine.

19. The vaccine of claim 18 wherein said vaccine is for nasal or parenteral administration.

20. The vaccine of claim 8 wherein said vaccine is for oral administration.

21. A method of expressing a polypeptide of Spike (S1) protein or functional fragment thereof of Severe Acute Respiratory Syndrome Coronavirus 2 (SARS-CoV-2) the method comprising, introducing into a plant a construct comprising,
(a) a promoter preferentially directing expression to seed tissue of a plant;
(b) a nucleic acid molecule encoding a S1 polypeptide of said SARS-CoV-2 as disclosed herein or a sequence having at least 90% identity thereto or a functional fragment operably linked to said promoter; and
(c) a nucleic acid molecule targeting expression of said polypeptide in the endoplasmic reticulum of said plant; and expressing said S1 polypeptide in said plant at levels of at least 1 mg/kg of seed of said plant.

22. The method of claim 21, wherein said construct further comprises a sequence selected from a sequence SEQ ID NO: 1, 2, 3, or 4 or a sequence with 90% homology thereto.

23. The method of claim 21, wherein said construct comprises two copies of said nucleic acid molecule encoding a S1 polypeptide.

24. The method of claim 21, wherein said construct comprises: pr44-BAASS:Cov-S1, pr44-BAASS:Cov-S1 KDEL, pr44-BAASS:Cov-S1-DC peptide, or pr44-BAASS:Cov-S1 LTB.

25. The method of claim 21, wherein said S1 protein or fragment thereof is expressed in said plant or plant product at levels of at least 10 mg/kg of whole seed.


SEQUENCE LISTING (24 sequences)

SEQ ID NO: 1 - DNA, 2115 bp, Artificial Sequence
  synthetic
ccatggctaataagcacctctcactctccctgttcctcgtcctcctcgggctctccgcctccctcgcctcgggggttcagctccctcctgcctacaccaactccttcacgcgcggcgtgtactaccccgacaaggtcttcaggtccagcgtgctgcactccacgcaggacctcttcctgccgttcttcagcaacgtgacctggttccacgccatccacgtcagcggcaccaacggcacgaagcgcttcgacaacccggtgctgcccttcaacgacggcgtctacttcgcgtccaccgagaagagcaacatcatcaggggctggatcttcggcaccacgctcgactccaagacgcagagcctcctgatcgtgaacaacgccaccaacgtggtcatcaaggtctgcgagttccagttctgcaatgaccccttcctgggcgtgtactaccacaagaacaacaagtcctggatggagagcgagttccgcgtgtactccagcgcgaacaactgcacgttcgagtatgtgtcccagccgttcctcatggacctggagggcaagcagggcaacttcaagaacctccgcgagttcgtcttcaagaacatcgacggctacttcaagatctactccaagcacaccccgatcaacctggtgagggacctcccacagggcttcagcgctctggagcccctggtggacctccccatcggcatcaacatcacccgcttccagacgctcctggctctgcacaggagctacctcaccccaggcgactcgtcctcgggctggacggccggcgcggccgcctactacgtgggctacctgcagccgcgcaccttcctcctgaagtacaacgagaacggcacgatcaccgacgccgtggactgcgctctggacccactctcggagaccaagtgcacgctgaagagcttcaccgtcgagaagggcatctaccagacgtccaacttccgcgtgcagccgaccgagagcatcgtcaggttcccgaacatcacgaacctctgccccttcggcgaggtgttcaacgcgaccaggttcgcctccgtctacgcctggaaccgcaagaggatctccaactgcgtcgccgactacagcgtgctctacaacagcgcgtccttcagcaccttcaagtgctacggcgtgtccccgacgaagctgaacgacctctgcttcaccaacgtctacgccgacagcttcgtgatccgcggcgacgaggtcaggcagatcgctccgggccagacgggcaagatcgccgactacaactacaagctgccggacgacttcaccggctgcgtgatcgcgtggaactccaacaacctcgacagcaaggtcggcggcaactacaactacctgtaccgcctcttcaggaagtccaacctgaagcccttcgagcgcgacatctccacggagatctaccaggcgggcagcaccccctgcaacggcgtggagggcttcaactgctacttcccgctccagagctacggcttccagccaaccaacggcgtgggctaccagccgtacagggtggtcgtgctctccttcgagctgctccacgcccccgctaccgtgtgcggccccaagaagagcaccaacctcgtcaagaacaagtgcgtgaacttcaacttcaacggcctgaccggcacgggcgtgctcaccgagtccaacaagaagttcctgcccttccagcagttcggcagggacatcgcggacacgaccgacgcggtcagggacccacagaccctggagatcctcgacatcaccccctgctccttcggcggcgtcagcgtgatcacccccggcaccaacacgagcaaccaggtcgcggtgctgtaccaggacgtgaactgcacggaggtccccgtggccatccacgctgaccagctgacgcccacctggagggtgtactccaccggcagcaacgtcttccagacgagggcgggctgcctcatcggcgccgagcatgtcaacaacagctacgagtgcgacatcccgatcggggcgggcatttgcgcttcctaccagacacagacgaactccccacggcgggcttgatagttaacctagacttgtccatcttctggattggccaacttaattaatg

SEQ ID NO: 2 - DNA, 2127 bp, Artificial Sequence
  synthetic
ccatggctaataagcatctgtctctctcactcttcctcgtcctcctcgggctctcagcgtctctcgcctcaggggtccagctcccaccggcctacaccaactccttcacgcgcggcgtgtactaccccgacaaggtcttcaggtccagcgtgctgcactccacgcaggacctcttcctgccgttcttcagcaacgtgacctggttccacgccatccacgtcagcggcaccaacggcacgaagcgcttcgacaacccggtgctgcccttcaacgacggcgtctacttcgcgtccaccgagaagagcaacatcatcaggggctggatcttcggcaccacgctcgactccaagacgcagagcctcctgatcgtgaacaacgccaccaacgtggtcatcaaggtctgcgagttccagttctgcaatgaccccttcctgggcgtgtactaccacaagaacaacaagtcctggatggagagcgagttccgcgtgtactccagcgcgaacaactgcacgttcgagtatgtgtcccagccgttcctcatggacctggagggcaagcagggcaacttcaagaacctccgcgagttcgtcttcaagaacatcgacggctacttcaagatctactccaagcacaccccgatcaacctggtgagggacctcccacagggcttcagcgctctggagcccctggtggacctccccatcggcatcaacatcacccgcttccagacgctcctggctctgcacaggagctacctcaccccaggcgactcgtcctcgggctggacggccggcgcggccgcctactacgtgggctacctgcagccgcgcaccttcctcctgaagtacaacgagaacggcacgatcaccgacgccgtggactgcgctctggacccactctcggagaccaagtgcacgctgaagagcttcaccgtcgagaagggcatctaccagacgtccaacttccgcgtgcagccgaccgagagcatcgtcaggttcccgaacatcacgaacctctgccccttcggcgaggtgttcaacgcgaccaggttcgcctccgtctacgcctggaaccgcaagaggatctccaactgcgtcgccgactacagcgtgctctacaacagcgcgtccttcagcaccttcaagtgctacggcgtgtccccgacgaagctgaacgacctctgcttcaccaacgtctacgccgacagcttcgtgatccgcggcgacgaggtcaggcagatcgctccgggccagacgggcaagatcgccgactacaactacaagctgccggacgacttcaccggctgcgtgatcgcgtggaactccaacaacctcgacagcaaggtcggcggcaactacaactacctgtaccgcctcttcaggaagtccaacctgaagcccttcgagcgcgacatctccacggagatctaccaggcgggcagcaccccctgcaacggcgtggagggcttcaactgctacttcccgctccagagctacggcttccagccaaccaacggcgtgggctaccagccgtacagggtggtcgtgctctccttcgagctgctccacgcccccgctaccgtgtgcggccccaagaagagcaccaacctcgtcaagaacaagtgcgtgaacttcaacttcaacggcctgaccggcacgggcgtgctcaccgagtccaacaagaagttcctgcccttccagcagttcggcagggacatcgcggacacgaccgacgcggtcagggacccacagaccctggagatcctcgacatcaccccctgctccttcggcggcgtcagcgtgatcacccccggcaccaacacgagcaaccaggtcgccgtgctgtaccaggacgtgaactgcacggaggtccccgtggccatccacgctgaccagctgacgcccacctggagggtgtactccaccggcagcaacgtcttccagacgagggcgggctgcctcatcggcgccgagcatgtcaacaacagctacgagtgcgacatcccgatcggcgcgggcatctgcgcttcataccagactcagacaaactctccacggcgggctaaggacgagctgtgatagttaacctagacttgtccatcttctggattggccaacttaattaatg

SEQ ID NO: 3 - DNA, 2151 bp, Artificial Sequence
  synthetic
ccatggctaataagcacctctccctgtctctcttcctcgtcctcctggggctctctgcctcgctcgcctctggggttcagctcccgcctgcctacaccaactccttcacgcgcggcgtgtactaccccgacaaggtcttcaggtccagcgtgctgcactccacgcaggacctcttcctgccgttcttcagcaacgtgacctggttccacgccatccacgtcagcggcaccaacggcacgaagcgcttcgacaacccggtgctgcccttcaacgacggcgtctacttcgcgtccaccgagaagagcaacatcatcaggggctggatcttcggcaccacgctcgactccaagacgcagagcctcctgatcgtgaacaacgccaccaacgtggtcatcaaggtctgcgagttccagttctgcaacgaccccttcctgggcgtgtactaccacaagaacaacaagtcctggatggagagcgagttccgcgtgtactccagcgcgaacaactgcacgttcgagtatgtgtcccagccgttcctcatggacctggagggcaagcagggcaacttcaagaacctccgcgagttcgtcttcaagaacatcgacggctacttcaagatctactccaagcacaccccgatcaacctggtgagggacctcccacagggcttcagcgctctggagcccctggtggacctccccatcggcatcaacatcacccgcttccagacgctcctggctctgcacaggagctacctcaccccaggcgactcgtcctcgggctggacggccggcgcggccgcctactacgtgggctacctgcagccgcgcaccttcctcctgaagtacaacgagaacggcacgatcaccgacgccgtggactgcgctctggacccactctcggagaccaagtgcacgctgaagagcttcaccgtcgagaagggcatctaccagacgtccaacttccgcgtgcagccgaccgagagcatcgtcaggttcccgaacatcacgaacctctgccccttcggcgaggtgttcaacgcgaccaggttcgcctccgtctacgcctggaaccgcaagaggatctccaactgcgtcgccgactacagcgtgctctacaacagcgcgtccttcagcaccttcaagtgctacggcgtgtccccgacgaagctgaacgacctctgcttcaccaacgtctacgccgacagcttcgtgatccgcggcgacgaggtcaggcagatcgctccgggccagacgggcaagatcgccgactacaactacaagctgccggacgacttcaccggctgcgtgatcgcgtggaactccaacaacctcgacagcaaggtcggcggcaactacaactacctgtaccgcctcttcaggaagtccaacctgaagcccttcgagcgcgacatctccacggagatctaccaggcgggcagcaccccctgcaacggcgtggagggcttcaactgctacttcccgctccagagctacggcttccagccaaccaacggcgtgggctaccagccgtacagggtggtcgtgctctccttcgagctgctccacgcccccgctaccgtgtgcggccccaagaagagcaccaacctcgtcaagaacaagtgcgtgaacttcaacttcaacggcctgaccggcacgggcgtgctcaccgagtccaacaagaagttcctgcccttccagcagttcggcagggacatcgcggacacgaccgacgcggtcagggacccacagaccctggagatcctcgacatcaccccctgctccttcggcggcgtcagcgtgatcacccccggcaccaacacgagcaaccaggtcgcggtgctgtaccaggacgtgaactgcacggaggtccccgtggccatccacgctgaccagctgacgcccacctggagggtgtactccaccggctccaacgtcttccagacgagggcgggctgcctcatcggcgccgagcacgtcaacaactcctacgagtgcgacatccccatcggcgcgggcatctgcgccagctaccagacccagacgaactcacccaggagggctttctacccgtcttaccatagcacaccccagcggccttgatgattaacctagacttgtccatcttctggattggccaacttaattaata

SEQ ID NO: 4 - DNA, 2424 bp, Artificial Sequence
  synthetic
ccatggctaataagcatctgtccctgtccctgttcctggtcctcctggggctctcagcgtccctcgcctccggggttcagctccctccagcctacaccaactccttcacgcgcggcgtgtactaccccgacaaggtcttcaggtccagcgtgctgcactccacgcaggacctcttcctgccgttcttcagcaacgtgacctggttccacgccatccacgtcagcggcaccaacggcacgaagcgcttcgacaacccggtgctgcccttcaacgacggcgtctacttcgcgtccaccgagaagagcaacatcatcaggggctggatcttcggcaccacgctcgactccaagacgcagagcctcctgatcgtgaacaacgccaccaacgtggtcatcaaggtctgcgagttccagttctgcaacgaccccttcctgggcgtgtactaccacaagaacaacaagtcctggatggagagcgagttccgcgtgtactccagcgcgaacaactgcaccttcgagtatgtgtcccagccgttcctcatggacctggagggcaagcagggcaacttcaagaacctccgcgagttcgtcttcaagaacatcgacggctacttcaagatctactccaagcacaccccgatcaacctggtgagggacctcccacagggcttcagcgctctggagcccctggtggacctccccatcggcatcaacatcacccgcttccagacgctcctggctctgcacaggagctacctcaccccaggcgactcgtcctcgggctggacggccggcgcggccgcctactacgtgggctacctgcagccgcgcaccttcctcctgaagtacaacgagaacggcacgatcaccgacgccgtggactgcgctctggacccactctcggagaccaagtgcacgctgaagagcttcaccgtcgagaagggcatctaccagacgtccaacttccgcgtgcagccgaccgagagcatcgtcaggttcccgaacatcacgaacctctgccccttcggcgaggtgttcaacgcgaccaggttcgcctccgtctacgcctggaaccgcaagaggatctccaactgcgtcgccgactacagcgtgctctacaacagcgcgtccttcagcaccttcaagtgctacggcgtctccccgacgaagctgaacgacctctgcttcaccaacgtctacgccgacagcttcgtgatccgcggcgacgaggtcaggcagatcgctccgggccagacgggcaagatcgccgactacaactacaagctgccggacgacttcaccggctgcgtgatcgcgtggaactccaacaacctcgacagcaaggtcggcggcaactacaactacctgtaccgcctcttcaggaagtccaacctgaagcccttcgagcgcgacatctccacggagatctaccaggcgggcagcaccccctgcaacggcgtggagggcttcaactgctacttcccgctccagagctacggcttccagccaaccaacggcgtgggctaccagccgtacagggtggtcgtgctctccttcgagctgctccacgcccccgctaccgtgtgcggccccaagaagagcaccaacctcgtcaagaacaagtgcgtgaacttcaacttcaacggcctgaccggcacgggcgtgctcaccgagtccaacaagaagttcctgcccttccagcagttcggcagggacatcgcggacacgaccgacgcggtcagggacccacagaccctggagatcctcgacatcaccccctgctccttcggcggcgtcagcgtgatcacccccggcaccaacacgagcaaccaggtcgcggtgctgtaccaggacgtgaactgcacggaggtccccgtggccatccacgctgaccagctgacgcccacctggagggtgtactccaccggctccaacgtcttccagacgagggcgggctgcctcatcggcgccgagcacgtcaacaactcctacgagtgcgacatccccatcggcgcgggcatctgcgcctcctaccagacgcagaccaacagcccacgcagggcggccccgcagtccatcacggagctgtgcagcgagtaccacaacacccagatctacacgatcaacgacaagatcctctcctacaccgagagcatggccggcaagagggagatggtcatcatcaccttcaagtcgggcgccaccttccaggtggaggtgcccggctcccagcacatcgacagccagaagaaggccatcgagcgcatgaaggacaccctgaggatcacgtacctcaccgagacgaagatcgacaagctctgcgtttggaacaacaagacacctaactctattgctgcgatttcgatggagaactgatgattaacctagacttgtccatcttctggattggccaacttaattaata

SEQ ID NO: 5 - DNA, 72 bp, Hordeum vulgare
atggcgaacaagcacctgagccttagcctcttcctcgtgctcctgggcctctccgcctccctcgcctccggc

SEQ ID NO: 6 - protein, 24 aa, Hordeum vulgare
MANKHLSLSLFLVLLGLSASLASG

SEQ ID NO: 7 - DNA, 36 bp, Artificial Sequence
  synthetic
ttctacccctcctaccacagcaccccacagcgcccc

SEQ ID NO: 8 - DNA, 309 bp, Escherichia coli
gccccgcagtccatcaccgagctctgctccgagtaccacaacacccagatctacaccatcaacgacaagatcctctcctacaccgagagcatggccggcaagcgcgagatggtgatcatcaccttcaagtccggcgccaccttccaggtggaggtgccgggctcccagcacatcgactcccagaagaaggccatcgagcgcatgaaggacaccctccgcatcacctacctcaccgagaccaagatcgacaagctctgcgtgtggaacaacaagaccccgaactccatcgccgccatcagcatggagaac

SEQ ID NO: 9 - protein, 103 aa, Escherichia coli
APQSITELCSEYHNTQIYTINDKILSYTESMAGKREMVIITFKSGATFQVEVPGSQHIDSQKKAIERMKDTLRITYLTETKIDKLCVWNNKTPNSIAAISMEN

SEQ ID NO: 10 - DNA, 3002 bp, Zea mays
cggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcaagcttgccgagtgccatccttggacactcgataaagtatattttatttttttttattttgccaaccaaactttttgtggtatgttcctacactatgtagatctacatgtaccattttggcacaattacaaaaatgttttctataactattagatttagttcgtttatttgaatttcttcggaaaattcacatatgaactgcaagtcactcgaaacatgaaaaaccgtgcatgcaaaataaatgatatgcatgttatctagcacaagttacgaccgaattcagaagcagaccagaatcttcaagcaccatgctcactaaacatgaccgtgaacttgttatccagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtattcagcaaagaagccgttgtcgatttactgttcgtcgagatctctttgccgagtgtcacactaggcaaagtctttacggagtgtttttcaggctttgacactcggcaaagcgctcgattccagtagtgacagtaatttgcatcaaaaatagccgagagatttaaaatgagtcaactaatagaccaactaattattagctattagtcgttagcttctttaatctaagctaaaaccaactaatagcttatttgttgaattacaattagctcaacggaattctctgttttttctataaaaaaaagggaaactgcccctcatttacagcaaactgtccgctgcctgtcgtccagatacaatgaacgtacctagtaggaactcttttacacgctcggtcgctcgccgcggatcggagtcccaggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaacacacacccgagcatatcacagtgacagacactacac

SEQ ID NO: 11 - DNA, 2520 bp, Zea mays
gcgctccctgacgctgtcttgggagagctgcaagatgagacactccatcccgcgcagccctgtcgtggcgtcctcctggatggacacctgcatcgctgtcgccctccaccaactcacctgaacgaagaatagaataaaaaatggagggagctgagggggcagtggttgcgctgtagggaggagagagaccgcgtcattataagactatctgcaaccgttacctctaaatttttccctctatatcattttttccccatattttcccccctattttttcatctcccgcaacggtttctcctaaatactccccctatatctcactaccactataaaatattattttttataccaactatcaattttttatctactaacaattactcgtggacccacagcacagtgtttaggagatgaacagtgacacgctatatctggggggagagagaaagaggccggcgcgtagggggcgccgtaggggcactgctgcggctgtagagtaccccctacacgccgcatgcaagggaagggggctgtcaggggggcaatgttgcgcatagcctaaagagcggatgaagcggcttgcaatttgcacgctggattcataaatagtgcatattactaaaaaaaagggtggggataggtatagagagtctattagagttgatctaagacccggtttatttcagattataatctgtccggattatataatccagcgcaaataatacagtaggtaaacaaacaactagattatgggttcagattatataatctaaaccccagattatgataatctcataatctcctcaagagtagcttattggagattattttggcaaaagacccactacccatggttatgtaaatagaaattataatatatatcatcttttttctcaccttaaataaacaaataagggtattgttgtctttatgaataatctacatttgtataatctaaactaccaaacaactacatctagattataatctggattatataatttaaattataatctagattatataatttataagctgaaacaacccggccctaaagcactatcgtatcacctatctgaaataagtcacgggtttcgaacgtccacttgcgtcgcacggaattgcatgtttcttgttggaagcatattcacgcaatctccacacataaaggtttatgtataaacttacatttagctcagtttaattacagtcttatttggatgcatatgtatggttctcaatccatataagttagagtaaaaaataagtttaaattttatcttaattcactccaacatatatggattgagtacaatactcatgtgcatccaaacaaactacttatattgaggtgaatttggatagaaattaaactaacttacacactaagccaatctttactatattaaagcaccagtttcaacgatcgtcccgcgtcaatattattaaaaaactcctacatttctttataatcaacccgcactcttataatctcttctctactactataataagagagtttatgtacaaaataaggtgaaattatgtataagtgttctggatattggttgttggctccatattcacacaacctaatcaatagaaaacatatgttttattaaaacaaaatttatcatatatcatatatatatatataaaccgtagcaatgcacgggcatataactagtgcaacttaatacatgtgtgtattaagatgaataagagggtatccaaataaaaaacttgttcgcttacgtctggatcgaaaggggttggaaacgattaaatctcttcctagtcaaaattgaatagaaggagatttaatctctcccaatccccttcgatcatccaggtgcaaccgtataagtcctaaagtggtgaggaacacgaaacaaccatgcattggcatgtaaagctccaagaatttgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacacc

SEQ ID NO: 12 - DNA, 6510 bp, Artificial Sequence
  synthetic
ggcgcgccggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcacgcgccggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcacgcgccggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcaagcttgccgagtgccatccttggacactcgataaagtatattttatttttttttattttgccaaccaaactttttgtggtatgttcctacactatgtagatctacatgtaccattttggcacaattacaaaaatgttttctataactattagatttagttcgtttatttgaatttcttcggaaaattcacatatgaactgcaagtcactcgaaacatgaaaaaccgtgcatgcaaaataaatgatatgcatgttatctagcacaagttacgaccgaattcagaagcagaccagaatcttcaagcaccatgctcactaaacatgaccgtgaacttgttatccagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtattcagcaaagaagccgttgtcgatttactgttcgtcgagatctctttgccgagtgtcacactaggcaaagtctttacggagtgtttttcaggctttgacactcggcaaagcgctcgattccagtagtgacagtaatttgcatcaaaaatagccgagagatttaaaatgagtcaactaatagaccaactaattattagctattagtcgttagcttctttaatctaagctaaaaccaactaatagcttatttgttgaattacaattagctcaacggaattctctgttttttctataaaaaaaagggaaactgcccctcatttacagcaaactgtccgctgcctgtcgtccagatacaatgaacgtacctagtaggaactcttttacacgctcggtcgctcgccgcggatcggagtcccaggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaacacacacccgagcatatcacagtgacagacactaca

SEQ ID NO: 13 - DNA, 12 bp, Artificial Sequence
  synthetic
aaggacgagctc

SEQ ID NO: 14 - protein, 4 aa, Artificial Sequence
  synthetic
HDEL

SEQ ID NO: 15 - DNA, 2217 bp, Artificial Sequence
  synthetic
atggcgaacaagcacctgagccttagcctcttcctcgtgctcctgggcctctccgcctccctcgcctccggcgtcactcgctgccagtcgaccatcaacttccgcaggttcttctccaagttcaacgtgcaggcccccgcggtggtggtgctcggcggctacctgccctccatgaattctagctcctggtactgcggcacgggcatcgagacggactcgggcgtgcacggcatcttcctcagctacatcgactccggccagggcttcgagatcggcatctcgcaggagcccttcgacccatcgggctaccagctctacctgcacaaggccaccaacggcaacacgaacgcgatcgcccgcctcaggatctgccagttcccggacaacaagaccctgggcccaaccgtcaacgacgtgaccaccggccgcaactgcctgttcaacaaggcgatcccggcctacctccaggacggcaagaacatcgtcgtgggcatcacgtgggacaacgacagggtcaccgtgttcgcggacaagatctaccacttctacctcaagaacgactggtcccgcgtcgcgaccaggtgctacaacaagcgcagctgcgccatgcagtatgtgtacaccccgacgtactacatgctcaacgtgacctcggcgggcgaggacggcatctactacgagccgtgcacggccaactgctcgggctacgcggccaacgtgttcgctaccgactccaacggccacatcccggagggcttcagcttcaacaactggttcctcctgagcaacgactccaccctcctgcacggcaaggtcgtgtccaaccagccgctcctggtcaactgcctcctggcgatccccaagatctacggcctgggccagttcttcagcttcaaccagacgatggacggcgtgtgcaacggcgcggccgctcagagggcccccgaggccctcaggttcaacatcaacgacacgagcgtcatcctggccgagggctccatcgtgctccacacggctctgggcacgaacctcagcttcgtgtgctccaacagctccgacccccacctggccatcttcgctatccccctgggcgccacggaggtcccctactactgcttcctgaaggtggacacctacaacagcacggtctacaagttcctggcggtgctgccgcccacggtcagggagatcgtgatcaccaagtacggcgacgtctacgtgaacggcttcggctacctccacctgggcctcctggacgcggtcaccatcaacttcaccggccacggcacggacgacgacgtgagcggcttctggacgatcgcgagcaccaacttcgtggacgctctcatcgaggtgcagggcaccgcgatccagaggatcctgtactgcgacgacccggtcagccagctcaagtgctcccaggtggccttcgacctggacgacggcttctaccccatcagctcccgcaacctcctgagccacgagcagccgatctccttcgtcaccctgcccagcttcaacgaccactccttcgtcaacatcacggtgagcgccgctttcggtggccactcgggcgccaacctcatcgcttccgacaccacgatcaacggcttcagctccttctgcgtggacacgcgccagttcaccatcacgctgttctacaacgtcaccaactcctacggctacgtgagcaagtcccaggactccaactgcccgttcaccctgcagagcgtcaacgactacctcagcttctccaagttctgcgtgagcacgtccctcctggccggcgcttgcaccatcgacctcttcggctaccccgagttcggcagcggcgtcaagttcacgtccctgtacttccagttcaccaagggcgagctgatcacgggcacccccaagccactggagggcgtcacggacgtgagcttcatgaccctcgacgtgtgcaccaagtacacgatctacggcttcaagggcgagggcatcatcaccctgacgaactcaagctttctcgccggcgtctactacaccagcgactccggccagctcctggcgttcaagaacgtgacctcgggcgcggtctactcggtgacgccctgcagcttctccgagcaggccgcctacgtggacgacgacatcgtcggcgtgatcagctccctgagcaactcaacattcaacaacactcgggagctgcctggcttcttctaccattcctgatag

SEQ ID NO: 16 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - protein, 687 aa, Artificial Sequence
  synthetic
MANKHLSLSLFLVLLGLSASLASGVQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRA

SEQ ID NO: 18 - protein, 691 aa, Artificial Sequence
  synthetic
MANKHLSLSLFLVLLGLSASLASGVQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAKDEL

SEQ ID NO: 19 - protein, 699 aa, Artificial Sequence
  synthetic
MANKHLSLSLFLVLLGLSASLASGVQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAFYPSYHSTPQRP

SEQ ID NO: 20 - protein, 790 aa, Artificial Sequence
  synthetic
MANKHLSLSLFLVLLGLSASLASGVQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAAPQSITELCSEYHNTQIYTINDKILSYTESMAGKREMVIITFKSGATFQVEVPGSQHIDSQKKAIERMKDTLRITYLTETKIDKLCVWNNKTPNSIAAISMEN

SEQ ID NO: 21 - protein, 663 aa, Artificial Sequence
  synthetic
VQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRA

SEQ ID NO: 22 - protein, 667 aa, Artificial Sequence
  synthetic
VQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAKDEL

SEQ ID NO: 23 - protein, 675 aa, Artificial Sequence
  synthetic
VQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAFYPSYHSTPQRP

SEQ ID NO: 24 - protein, 766 aa, Artificial Sequence
  synthetic
VQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAAPQSITELCSEYHNTQIYTINDKILSYTESMAGKREMVIITFKSGATFQVEVPGSQHIDSQKKAIERMKDTLRITYLTETKIDKLCVWNNKTPNSIAAISMEN